SEQUENCE LISTING (115 sequences)

SEQ ID NO: 1 - DNA, 64 bp, ARTIFICIAL SEQUENCE
  synthetic
gggagataggtagtttcatgttgttgggcctgggtttctgaacacaacaacattaaaccacccg

SEQ ID NO: 2 - DNA, 62 bp, ARTIFICIAL SEQUENCE
  synthetic
gggagataggtagtttcctgttgttgggatccacctttctctcgacagcacgacactgcctt

SEQ ID NO: 3 - DNA, 65 bp, ARTIFICIAL SEQUENCE
  synthetic
gggagattcaagtaatccaggataggctgtgcaggtcccaatgggcctattcttggttacttgca

SEQ ID NO: 4 - DNA, 63 bp, ARTIFICIAL SEQUENCE
  synthetic
gggagaaacccgtagatccgatcttgtggtgaagtggaccgcacaagctcgcttctatgggtc

SEQ ID NO: 5 - DNA, 23 bp, ARTIFICIAL SEQUENCE
  synthetic
cggcctgattcacaacaccagct

SEQ ID NO: 6 - DNA, 22 bp, ARTIFICIAL SEQUENCE
  synthetic
ggtaagaggatgcgctgctcgt

SEQ ID NO: 7 - DNA, 23 bp, ARTIFICIAL SEQUENCE
  synthetic
acaaacaccattgtcacactcca

SEQ ID NO: 8 - DNA, 17 bp, ARTIFICIAL SEQUENCE
  synthetic
gattcacaacaccagct

SEQ ID NO: 9 - DNA, 22 bp, ARTIFICIAL SEQUENCE
  synthetic
ggtaagaggatgcgctgctcgt

SEQ ID NO: 10 - DNA, 23 bp, ARTIFICIAL SEQUENCE
  synthetic
acaaacaccattgtcacactcca

SEQ ID NO: 11 - DNA, 17 bp, ARTIFICIAL SEQUENCE
  synthetic
gattcacaacaccagct

SEQ ID NO: 12 - DNA, 22 bp, ARTIFICIAL SEQUENCE
  synthetic
ggtaagaggatgcgctgctcgt

SEQ ID NO: 13 - DNA, 25 bp, ARTIFICIAL SEQUENCE
  synthetic
ttctttacattccatagcttagcag

SEQ ID NO: 14 - DNA, 25 bp, ARTIFICIAL SEQUENCE
  synthetic
acttctttacattccatagcattgt

SEQ ID NO: 15 - DNA, 23 bp, ARTIFICIAL SEQUENCE
  synthetic
atggcgtttgatagtttagacac

SEQ ID NO: 16 - DNA, 16 bp, ARTIFICIAL SEQUENCE
  synthetic
caccgcgtgccttaat

SEQ ID NO: 17 - DNA, 24 bp, ARTIFICIAL SEQUENCE
  synthetic
ttgtatgacattaaatcaaggtcc

SEQ ID NO: 18 - DNA, 24 bp, ARTIFICIAL SEQUENCE
  synthetic
gcgtgccttaattgtatagacatt

SEQ ID NO: 19 - DNA, 22 bp, ARTIFICIAL SEQUENCE
  synthetic
tccgatgatctttctgaatcag

SEQ ID NO: 20 - DNA, 22 bp, ARTIFICIAL SEQUENCE
  synthetic
cactgtgagaaatgtaaacctc

SEQ ID NO: 21 - DNA, 20 bp, ARTIFICIAL SEQUENCE
  synthetic
aagggcttcctgactactgt

SEQ ID NO: 22 - DNA, 17 bp, ARTIFICIAL SEQUENCE
  synthetic
ggcttccggctattgag

SEQ ID NO: 23 - DNA, 17 bp, ARTIFICIAL SEQUENCE
  synthetic
cattgcactgcttccca

SEQ ID NO: 24 - DNA, 17 bp, ARTIFICIAL SEQUENCE
  synthetic
cacagggtgaacacagt

SEQ ID NO: 25 - DNA, 17 bp, ARTIFICIAL SEQUENCE
  synthetic
tctgattggcaacggcc

SEQ ID NO: 26 - DNA, 15 bp, ARTIFICIAL SEQUENCE
  synthetic
cgggtaagaggatgc

SEQ ID NO: 27 - DNA, 20 bp, ARTIFICIAL SEQUENCE
  synthetic
agaagcatttccacacactg

SEQ ID NO: 28 - DNA, 17 bp, ARTIFICIAL SEQUENCE
  synthetic
ggcctgcaccttttcaa

SEQ ID NO: 29 - DNA, 27 bp, ARTIFICIAL SEQUENCE
  synthetic
agataattttagaatcttaacgccaat

SEQ ID NO: 30 - DNA, 23 bp, ARTIFICIAL SEQUENCE
  synthetic
agtaatattggtgtttaatatat

SEQ ID NO: 31 - DNA, 20 bp, ARTIFICIAL SEQUENCE
  synthetic
gtccaacacttacagtcaca

SEQ ID NO: 32 - DNA, 12 bp, ARTIFICIAL SEQUENCE
  synthetic
tggggaggggac

SEQ ID NO: 33 - DNA, 13 bp, ARTIFICIAL SEQUENCE
  synthetic
cagagcagcgccc

SEQ ID NO: 34 - DNA, 20 bp, ARTIFICIAL SEQUENCE
  synthetic
tgttgtgttcagaaacccag

SEQ ID NO: 35 - DNA, 23 bp, ARTIFICIAL SEQUENCE
  synthetic
agtttcttgttgccgagttcaaa

SEQ ID NO: 36 - DNA, 18 bp, ARTIFICIAL SEQUENCE
  synthetic
cgagagaaaggtggatcc

SEQ ID NO: 37 - DNA, 27 bp, ARTIFICIAL SEQUENCE
  synthetic
tatagagaagaaggaaaaatcacagga

SEQ ID NO: 38 - DNA, 18 bp, ARTIFICIAL SEQUENCE
  synthetic
cacattgagagcctcctg

SEQ ID NO: 39 - DNA, 20 bp, ARTIFICIAL SEQUENCE
  synthetic
aacggcattgtcctgaacag

SEQ ID NO: 40 - DNA, 23 bp, ARTIFICIAL SEQUENCE
  synthetic
ttgcacaactacattcttcttgt

SEQ ID NO: 41 - DNA, 17 bp, ARTIFICIAL SEQUENCE
  synthetic
acagctggatgcaaacc

SEQ ID NO: 42 - DNA, 25 bp, ARTIFICIAL SEQUENCE
  synthetic
tacatatatacgctgaaatgcaaac

SEQ ID NO: 43 - DNA, 19 bp, ARTIFICIAL SEQUENCE
  synthetic
gcagtattagagactccca

SEQ ID NO: 44 - DNA, 20 bp, ARTIFICIAL SEQUENCE
  synthetic
ctttaggccagatcctcaaa

SEQ ID NO: 45 - DNA, 20 bp, ARTIFICIAL SEQUENCE
  synthetic
tcacaattgcgctacagaac

SEQ ID NO: 46 - DNA, 20 bp, ARTIFICIAL SEQUENCE
  synthetic
gccatgagattcaacagtca

SEQ ID NO: 47 - DNA, 17 bp, ARTIFICIAL SEQUENCE
  synthetic
ggtgagcggatgttctg

SEQ ID NO: 48 - DNA, 17 bp, ARTIFICIAL SEQUENCE
  synthetic
ttcctgctgaactgagc

SEQ ID NO: 49 - DNA, 23 bp, ARTIFICIAL SEQUENCE
  synthetic
tgtacacaaaccaactgtgtttc

SEQ ID NO: 50 - DNA, 15 bp, ARTIFICIAL SEQUENCE
  synthetic
cattgggacctgcac

SEQ ID NO: 51 - DNA, 18 bp, ARTIFICIAL SEQUENCE
  synthetic
cctcacagatggaaacag

SEQ ID NO: 52 - DNA, 18 bp, ARTIFICIAL SEQUENCE
  synthetic
cagctttacaccaatgcc

SEQ ID NO: 53 - DNA, 27 bp, ARTIFICIAL SEQUENCE
  synthetic
ctcaataaacaaattatgtcacaaaca

SEQ ID NO: 54 - DNA, 16 bp, ARTIFICIAL SEQUENCE
  synthetic
atctgagcaccacccg

SEQ ID NO: 55 - DNA, 19 bp, ARTIFICIAL SEQUENCE
  synthetic
ggctgtatgcactttctcc

SEQ ID NO: 56 - DNA, 19 bp, ARTIFICIAL SEQUENCE
  synthetic
ctggtttgatctgggatcc

SEQ ID NO: 57 - DNA, 16 bp, ARTIFICIAL SEQUENCE
  synthetic
gtcgcctgagtcatca

SEQ ID NO: 58 - DNA, 18 bp, ARTIFICIAL SEQUENCE
  synthetic
tgcctccttcagagcaac

SEQ ID NO: 59 - DNA, 17 bp, ARTIFICIAL SEQUENCE
  synthetic
cgtgagctgtgtaactg

SEQ ID NO: 60 - DNA, 21 bp, ARTIFICIAL SEQUENCE
  synthetic
cccagtacagacaaaaaggaa

SEQ ID NO: 61 - DNA, 17 bp, ARTIFICIAL SEQUENCE
  synthetic
acaaaacgctcaggtcc

SEQ ID NO: 62 - DNA, 25 bp, ARTIFICIAL SEQUENCE
  synthetic
tccatgtcaataaaccgaatataca

SEQ ID NO: 63 - DNA, 22 bp, ARTIFICIAL SEQUENCE
  synthetic
tgattcgtcataagtaaagcga

SEQ ID NO: 64 - DNA, 17 bp, ARTIFICIAL SEQUENCE
  synthetic
agcaacattcgtcgtcc

SEQ ID NO: 65 - DNA, 25 bp, ARTIFICIAL SEQUENCE
  synthetic
ttttggaaaatagaaaagtctcgct

SEQ ID NO: 66 - DNA, 22 bp, ARTIFICIAL SEQUENCE
  synthetic
ccacgtaaggaaatagaggatc

SEQ ID NO: 67 - DNA, 16 bp, ARTIFICIAL SEQUENCE
  synthetic
acacagaggatctagc

SEQ ID NO: 68 - DNA, 22 bp, ARTIFICIAL SEQUENCE
  synthetic
actgagacatagttacaaagtc

SEQ ID NO: 69 - DNA, 20 bp, ARTIFICIAL SEQUENCE
  synthetic
tgactcgctttgatgaatcg

SEQ ID NO: 70 - DNA, 15 bp, ARTIFICIAL SEQUENCE
  synthetic
tggcgttctcaatgg

SEQ ID NO: 71 - DNA, 27 bp, ARTIFICIAL SEQUENCE
  synthetic
tgtttcatcataaataaagagaagaca

SEQ ID NO: 72 - DNA, 19 bp, ARTIFICIAL SEQUENCE
  synthetic
gaggtgcttttcatccagt

SEQ ID NO: 73 - DNA, 18 bp, ARTIFICIAL SEQUENCE
  synthetic
attcagacagcactctca

SEQ ID NO: 74 - DNA, 15 bp, ARTIFICIAL SEQUENCE
  synthetic
cccagagcaccgatc

SEQ ID NO: 75 - DNA, 24 bp, ARTIFICIAL SEQUENCE
  synthetic
gtttcaatcacacctaattacatg

SEQ ID NO: 76 - DNA, 18 bp, ARTIFICIAL SEQUENCE
  synthetic
actttcattctggcacca

SEQ ID NO: 77 - DNA, 20 bp, ARTIFICIAL SEQUENCE
  synthetic
acagcactttcattctggca

SEQ ID NO: 78 - DNA, 26 bp, ARTIFICIAL SEQUENCE
  synthetic
ggtgaaatttatattttagttcacat

SEQ ID NO: 79 - DNA, 20 bp, ARTIFICIAL SEQUENCE
  synthetic
actctgctttcagacaacag

SEQ ID NO: 80 - DNA, 26 bp, ARTIFICIAL SEQUENCE
  synthetic
agcactttcttttctttcagacaaca

SEQ ID NO: 81 - DNA, 26 bp, ARTIFICIAL SEQUENCE
  synthetic
gaagcactttctttcacaaaacacaa

SEQ ID NO: 82 - DNA, 23 bp, ARTIFICIAL SEQUENCE
  synthetic
ggaagcactttcttttctttcac

SEQ ID NO: 83 - DNA, 25 bp, ARTIFICIAL SEQUENCE
  synthetic
gctttctcttcttttagacaacaga

SEQ ID NO: 84 - DNA, 23 bp, ARTIFICIAL SEQUENCE
  synthetic
agcgctttgttttctttcagaca

SEQ ID NO: 85 - DNA, 25 bp, ARTIFICIAL SEQUENCE
  synthetic
agcgctttcttttcttttagacaag

SEQ ID NO: 86 - DNA, 25 bp, ARTIFICIAL SEQUENCE
  synthetic
tgtttaagagaaaacaaacagaaag

SEQ ID NO: 87 - DNA, 26 bp, ARTIFICIAL SEQUENCE
  synthetic
cactttgttttctttcaaacagaaag

SEQ ID NO: 88 - DNA, 25 bp, ARTIFICIAL SEQUENCE
  synthetic
cgccttcttttttttcacataagag

SEQ ID NO: 89 - DNA, 24 bp, ARTIFICIAL SEQUENCE
  synthetic
cgctttctcttctttcaagcaaca

SEQ ID NO: 90 - DNA, 25 bp, ARTIFICIAL SEQUENCE
  synthetic
tgcatgttcttttctttcaacagaa

SEQ ID NO: 91 - DNA, 26 bp, ARTIFICIAL SEQUENCE
  synthetic
aagcactttctcttctttcagacaac

SEQ ID NO: 92 - DNA, 25 bp, ARTIFICIAL SEQUENCE
  synthetic
actttcttttcttttagacaacaga

SEQ ID NO: 93 - DNA, 18 bp, ARTIFICIAL SEQUENCE
  synthetic
agactccacaccactcat

SEQ ID NO: 94 - DNA, 23 bp, ARTIFICIAL SEQUENCE
  synthetic
atgaagaccaatacactcataca

SEQ ID NO: 95 - DNA, 17 bp, ARTIFICIAL SEQUENCE
  synthetic
ttatgacggctctgtgg

SEQ ID NO: 96 - DNA, 20 bp, ARTIFICIAL SEQUENCE
  synthetic
aggtccacttcaccacaaga

SEQ ID NO: 97 - DNA, 76 bp, ARTIFICIAL SEQUENCE
  synthetic
tcagttttgcatagatttgcacaactacattcttcttgtagtgcaactatgcaaaacctatagtgagtcgtattaa

SEQ ID NO: 98 - DNA, 87 bp, ARTIFICIAL SEQUENCE
  synthetic
aaccctggtgtcgtgaaatagccgggtaagaggatgcgctgctcgtcggcctgattcacaacaccagcctatagtgagtcgtattaa

SEQ ID NO: 99 - DNA, 76 bp, ARTIFICIAL SEQUENCE; identical to SEQ ID NO: 97
  synthetic

SEQ ID NO: 100 - DNA, 87 bp, ARTIFICIAL SEQUENCE; identical to SEQ ID NO: 98
  synthetic

SEQ ID NO: 101 - DNA, 16 bp, ARTIFICIAL SEQUENCE
  synthetic
attgggacctgcacag

SEQ ID NO: 102 - DNA, 16 bp, ARTIFICIAL SEQUENCE
  synthetic
cctcacagatggaaac

SEQ ID NO: 103 - DNA, 15 bp, ARTIFICIAL SEQUENCE
  synthetic
aatggagaacaggct

SEQ ID NO: 104 - DNA, 21 bp, ARTIFICIAL SEQUENCE
  synthetic
gcaccttttcaaaatccacaa

SEQ ID NO: 105 - DNA, 19 bp, ARTIFICIAL SEQUENCE
  synthetic
tcgcatcttgactgtagca

SEQ ID NO: 106 - DNA, 21 bp, ARTIFICIAL SEQUENCE
  synthetic
tgaccacaaaattccttacac

SEQ ID NO: 107 - DNA, 21 bp, ARTIFICIAL SEQUENCE
  synthetic
tgactatacggataccacaca

SEQ ID NO: 108 - DNA, 15 bp, ARTIFICIAL SEQUENCE
  synthetic
gcggtccacttcacc

SEQ ID NO: 109 - DNA, 15 bp, ARTIFICIAL SEQUENCE
  synthetic
cttgtgtgcggcgaa

SEQ ID NO: 110 - DNA, 66 bp, ARTIFICIAL SEQUENCE
  synthetic
gggagaaccctgtagatccgaatttgtgtaaggaattttgtggtcacaaattcgtatctaggggaa

SEQ ID NO: 111 - DNA, 63 bp, ARTIFICIAL SEQUENCE
  synthetic
gggagatagcagcacataatggtttgtggattttgaaaaggtgcaggccatattgtgctgcct

SEQ ID NO: 112 - DNA, 65 bp, ARTIFICIAL SEQUENCE; identical to SEQ ID NO: 3
  synthetic

SEQ ID NO: 113 - DNA, 63 bp, ARTIFICIAL SEQUENCE; identical to SEQ ID NO: 4
  synthetic

SEQ ID NO: 114 - RNA, 62 nt, Artificial Sequence
  synthetic
agcugguguugugaaucaggccguugccaaucagagaacggcuacuucacgacaccagggcc

SEQ ID NO: 115 - RNA, 69 nt, Artificial Sequence
  synthetic
agcugguguugugaaucaggccgacgagcagcgcauccucuuacccggcuauuucacgacaccaggguu

(19) United States
(12) Patent Application Publication
(10) Pub. No.: US 2009/0023221 A1
(43) Pub. Date: Jan. 22, 2009

(54) Oligonucleotide probes useful for detection and analysis of microrna precursors

(75) Inventors: Gregor Obernosterer, Vienna (AT); Nina Stahlberg, Horsholm (DK); Soren Moller, Holte (DK); Soren M. Echwald, Humlebaek (DK); Peter Mouritzen, Jyllinge (DK); Peter S. Nielsen, Birkerod (DK); Mikkel Norholm, Belmont, MA (US); Kim Bundvig Barken, Jyllinge (DK)

Correspondence Address: CLARK & ELBING LLP, 101 FEDERAL STREET, BOSTON, MA, 02110, US

(73) Assignee: Exigon A/S, Vedbaek (DK)

(21) Appl. No.: 11/803,891

(22) Filed: May 16, 2007

Related U.S. Application Data
(60) Provisional application No. 60/801,839, filed on May 19, 2006.

(51) Int. Cl. G01N 33/00; C07H 21/04

(52) U.S. Cl. 436/94; 536/24.32

(57) ABSTRACT

The invention relates to ribonucleic acids and oligonucleotide probes useful for detection and analysis of microRNA precursors and their targets. The invention furthermore relates to oligonucleotide probes for detection and analysis of other non-coding RNAs, mRNAs, mRNA splice variants, allelic variants of single transcripts, mutations, deletions, or duplications of particular exons in transcripts, e.g., alterations associated with human disease, such as cancer.

### CROSS-REFERENCE TO RELATED APPLICATIONS

This application claims benefit of U.S. Provisional Application No. 60/801,839, filed May 19, 2006, which is hereby incorporated by reference.

### BACKGROUND OF THE INVENTION

The present invention relates to ribonucleic acids and oligonucleotide probes useful for detection and analysis of microRNA precursors. The invention furthermore relates to oligonucleotide probes for detection and analysis of targets of miRNA precursors, including other non-coding RNAs such as siRNAs, as well as mRNAs, mRNA splice variants, allelic variants of single transcripts, mutations, deletions, or duplications of particular exons in transcripts, e.g., alterations associated with human disease, such as cancer, and proteins that bind miRNA precursors.

The present invention relates to the detection and analysis of target nucleotide sequences in a wide variety of nucleic acid samples and more specifically to the methods employing the design and use of oligonucleotide probes that are useful for detecting and analyzing target nucleotide sequences, such as microRNA precursors, and proteins that bind microRNA precursors, for detecting differences between samples (e.g., such as samples from a cancer patient and a healthy patient).

Non-Coding RNAs

The expanding inventory of international sequence databases and the concomitant sequencing of nearly 200 genomes representing all three domains of life—bacteria, archea and eukaryota—have been the primary drivers in the process of deconstructing living organisms into comprehensive molecular catalogs of genes, transcripts and proteins. The importance of the genetic variation within a single species has become apparent, extending beyond the completion of genetic blueprints of several important genomes, culminating in the publication of the working draft of the human genome sequence in (Lander et al., Nature 409:860-921, 2001; Venter et al., Science 291:1304-1351, 2001; Sachidanandam et al., Nature 409:928-933, 2001). On the other hand, the increasing number of detailed, large-scale molecular analyses of transcription originating from the human and mouse genomes along with the recent identification of several types of non-protein-coding RNAs, such as small nuclear RNAs, siRNAs, pri-microRNAs, pre-microRNAs (microRNA precursors), microRNAs, and antisense RNAs, indicate that the transcriptomes of higher eukaryotes are much more complex than originally anticipated (Wong et al., Genome Research 11:1975-1977, 2001; Kampa et al., Genome Research 14:331-342, 2004).

RNA-mediated gene regulation is widespread in higher eukaryotes and complex genetic phenomena like RNA interference, co-suppression, transgene silencing, imprinting, methylation, and possibly position-effect variegation and transvection, all involve intersecting pathways based on or connected to RNA signaling (Mattick, EMBO reports 2, 11:986-991, 2001). Recent studies indicate that antisense transcription is a very common phenomenon in the mouse and human genomes (Okazaki et al., Nature Biotechnol. 420:563-573, 2003). Thus, antisense modulation of gene expression in eukaryotic cells, e.g., human cells appear to be a common regulatory mechanism.

As a result of the Central Dogma: ‘DNA makes RNA, and RNA makes protein’, RNAs have been considered as simple molecules that just translate the genetic information into protein. Recently, it has been estimated that although most of the genome is transcribed, almost 97% of the genome does not encode proteins in higher eukaryotes, but putative, non-coding RNAs (Wong et al., Genome Research 11:1975-1977, 2001). The non-coding RNAs (ncRNAs) appear to be particularly well suited for regulatory roles that require highly specific nucleic acid recognition. Therefore, the view of RNA is rapidly changing from merely its role as an informational molecule to a molecule having a wide variety of structural, informational and catalytic molecules in the cell.

MicroRNAs

Recently, a large number of small non-coding RNA genes have been identified and designated as microRNAs (miRNAs) for review, see (Ke et al., Curr. Opin. Chem. Biol. 7:516-523, 2003). miRNAs are a family of small (˜22-nt), endogenous, non-coding RNAs which, by binding complementary sequences in the 3′-untranslated region (3′-UTR) of messenger RNAs (mRNAs), either mediate translational repression or direct mRNA cleavage (Pillai, RNA, 11:1753-1761, 2005). The first miRNAs to be discovered were the lin-4 and let-7 that are heterochronic switching genes essential for the normal temporal control of diverse developmental events in the roundworm C. elegans (Lee et al., Cell 75:843-854, 1993; Reinhart et al., Nature 403:901-906, 2000). miRNAs have been evolutionarily conserved over a wide range of species and exhibit diversity in expression profiles, suggesting that they occupy a wide variety of regulatory functions and exert significant effects on cell growth and development (Ke et al., Curr. Opin. Chem. Biol. 7:516-523, 2003). miRNAs are thought to interact with target mRNAs by limited complementary and suppressed translation (Lagos-Quintana et al., Science 294:853-858, 2001; Lee and Ambros, Science 294:858-862, 2001). Many studies have shown, however, that perfect complementarity between miRNAs and their target RNA could lead to target RNA degradation rather than inhibiting translation (Hutvagner and Zamore, Science 297:2056-2060, 2002), suggesting that the degree of complementarity determines their functions. By identifying sequences with near complementarity, several targets have been predicted, most of which appear to be potential transcriptional factors that are crucial in cell growth and development. The high percentage of predicted miRNA targets acting as developmental regulators and the conservation of target sites suggest that miRNAs are involved in a wide range of organism development and behavior and cell fate decisions (Ke et al., Curr. Opin. Chem. Biol. 7:516-523, 2003).

More than 1345 microRNAs have been identified in humans, worms, fruit flies and plants according to the miRNA registry database release 5.0 in September 2004, hosted by Sanger Institute, UK, and many miRNAs that correspond to putative genes have also been identified. Some miRNAs have multiple loci in the genome (Reinhart et al., Genes Dev. 16:1616-1626, 2002) and occasionally, several miRNA genes are arranged in tandem clusters (Lagos-Quintana et al., Science 294:853-858, 2001). Of the miRNAs discovered thus far, more than 800 miRNAs have been experimentally discovered in mammals (Lagos-Quintana et al., Curr. Biol. 12:735-739, 2002; Bentwich et al., Nat. Genet. 37:766-770, 2005), and some of them are highly conserved between invertebrates and vertebrates (Bartel, Cell, 116:281-297, 2004). However, through the development of increasingly sophisticated algorithms based on machine learning techniques we are already considering thousands of predicted miRNAs (John et al., PLoS Biology 2:e363, 2004; Wang et al., Genome Biology 5:R65, 2004; Lim et al., Science 299:1540, 2003; Bentwich et al., Nat. Genet. 37:766-770, 2005). The fact that many of the miRNAs reported to date have been isolated just once suggests that many new miRNAs will be discovered in the future. A recent in-depth transcriptional analysis of the human chromosomes 21 and 22 found that 49% of the observed transcription was outside of any known annotation, and furthermore, that these novel transcripts were both coding and non-coding RNAs (Kampa et al., Genome Research 14:331-342, 2004). The identified miRNAs to date represent most likely the tip of the iceberg, and the number of miRNAs might turn out to be very large.

MicroRNA Precursors

miRNAs are transcribed as mono- or poly-cistronic, long, primary precursor transcripts (pri-miRNAs) that are cleaved into ˜70-nt precursor hairpins, known as microRNA precursors (pre-miRNAs), by the nuclear RNase III-like enzyme Drosha (Lee et al., Nature 425:415-419, 2003). MicroRNA precursors (pre-miRNAs) form hairpins having a loop region and a stem region containing a duplex of the opposite ends of the RNA strand. Subsequently pre-miRNA hairpins are exported to the cytoplasm by Exportin-5 (Yi et al., Genes & Dev., 17:3011-3016, 2003; Bohnsack et al., RNA, 10:185-191, 2004), where they are processed by a second RNase III-like enzyme, termed Dicer, into ˜22-nt duplexes (Bernstein et al., Nature 409:363-366, 2001), followed by the asymmetric assembly of one of the two strands into a functional miRNP or miRISC (Khvorova et al., Cell 115:209-216, 2003). miRNAs can recognize regulatory targets while part of the miRNP complex and inhibit protein translation. Alternatively, the active RISC complex is guided to degrade the specific target mRNAs (Lipardi et al., Cell 107:297-307, 2001; Zhang et al., EMBO J. 21:5875-5885, 2002; Nykanen et al., Cell 107:309-321, 2001). There are several similarities between miRNP and the RNA-induced silencing complex, miRISC, including similar sizes and both containing RNA helicase and the PPD proteins. It has therefore been proposed that miRNP and miRISC are the same RNP with multiple functions (Ke et al., Curr. Opin. Chem. Biol. 7:516-523, 2003).

Most reports in the literature have described the processing of miRNAs to be complete, suggesting that intermediates like pri-miRNA and pre-miRNA rarely accumulate in cells and tissues. However, previous studies describing miRNA profiles of cells and tissues have only investigated size-fractionated RNAs pools. Consequently the presence of larger miRNA precursors has been overlooked.

MicroRNAs, their Precursors, and Human Disease

mRNAs and their precursors may play important roles in human development and disease. Several human diseases have already been pinpointed in which miRNAs or their processing machinery might be implicated. One of them is spinal muscular atrophy (SMA), a pediatric neurodegenerative disease caused by reduced protein levels or loss-of-function mutations of the survival of motor neurons (SMN) gene (Paushkin et al., Curr. Opin. Cell Biol. 14:305-312, 2002). Two proteins (Gemin3 and Gemin4) that are part of the SMN complex are also components of miRNPs, whereas it remains to be seen whether miRNA biogenesis or function is dysregulated in SMA and what effect this has on pathogenesis. Another neurological disease linked to mi/siRNAs is fragile X mental retardation (FXMR) caused by absence or mutations of the fragile X mental retardation protein (FMRP) (Nelson et al., TIBS 28:534-540, 2003), and there are additional clues that miRNAs might play a role in other neurological diseases. Yet another interesting finding is that the miR-224 gene locus lies within the minimal candidate region of two different neurological diseases: early-onset Parkinsonism and X-linked mental retardation (Dostie et al., RNA 9:180-186, 2003). Links between cancer and miRNAs have also been recently described. The most frequent single genetic abnormality in chronic lymphocytic leukaemia (CLL) is a deletion localized to chromosome 13q14 (50% of the cases). A recent study determined that two different miRNA (miR15 and miR16) genes are clustered and located within the intron of LEU2, which lies within the deleted minimal region of the B-cell chronic lymphocytic leukaemia (B-CLL) tumour suppressor locus, and both genes are deleted or down-regulated in the majority of CLL cases (Calin et al., Proc. Natl. Acad. Sci. U.S.A. 99:15524-15529, 2002).

Alterations in miRNA biogenesis resulting in different levels of mature miRNAs and their miRNA precursors could illuminate the mechanisms underlying many disease processes. For example, the 26 miRNA precursors were equally expressed in non-cancerous and cancerous colorectal tissues from patients, whereas the expression of mature human miR143 and miR145 was greatly reduced in cancer tissues compared with non-cancer tissues, suggesting altered processing for specific miRNAs in human disease (Michael et al., Mol. Cancer. Res. 1:882-891, 2003).

Connections between miRNAs, their precursors, and human diseases will only strengthen in parallel with the knowledge of miRNA, their precursors, and the gene networks that they control. Moreover, the understanding of the regulation of RNA-mediated gene expression is leading to the development of novel therapeutic approaches that will be likely to revolutionize the practice of medicine (Nelson et al., TIBS 28:534-540, 2003).

Low-Throughput Analysis of microRNAs and microRNA Precursors

Many techniques have been employed to study miRNAs and their miRNA precursors. First, the research groups have used the small size of the miRNAs as a primary criterion for miRNA isolation and detection. In comparison, standard cDNA libraries would lack miRNAs, primarily because small RNAs are normally excluded by size selection in the cDNA library construction procedure. Total RNA from fly embryos, worms or HeLa cells has been size fractionated so that only molecules 25 nucleotides or smaller would be captured (Moss, Curr. Biology 12:R138-R140, 2002). Synthetic oligomers have then been ligated directly to the RNA pools using T4 RNA ligase, followed by reverse-transcription, amplification by PCR, cloning and sequencing of the ligated RNA (Moss, Curr. Biology 12:R138-R140, 2002; Grad et al., Mol. Cell. 11: 1253-1263, 2003). The genome databases have subsequently been queried with the sequences, confirming the origin of the miRNAs from these organisms as well as placing the miRNA genes physically in the context of other genes in the genome. The vast majority of the cloned sequences have been located in intronic regions or between genes, occasionally in clusters, suggesting that the tandemly arranged miRNAs are processed from a single transcript, pri-miRNA, to allow coordinate regulation. Furthermore, the genomic sequences have revealed the fold-back structures of the miRNA precursors (pre-miRNA) (Moss, Curr. Biology 12:R138-R140, 2002).

The size and low level of expression of many miRNAs require the use of sensitive and quantitative analysis tools. Due to their small size of 19-25 nt, the use of quantitative real-time PCR for monitoring expression of mature miRNAs is excluded; however, (Schmittgen et al., Nucleic Acids Res. 32:e43, 2004) successfully applied real-time PCR assays to quantify the expression of miRNA precursors. Most miRNA researchers currently use Northern blot analysis combined with polyacrylamide gels to examine expression of both the mature and precursor miRNAs (Reinhart et al., Nature 403:901-906, 2000; Lagos-Quintana et al., Science 294:853-858, 2001; Lee et al., Science 294:862-864, 2001). Primer extension has also been used to detect the mature miRNA (Zeng and Cullen, RNA 9:112-123, 2003). The primary disadvantages of all the gel-based assays (Northern blotting, primer extension, RNase protection assays etc.) as tools for monitoring miRNA expression is that these techniques are low-throughput and have poor sensitivity. Consequently, a large amount of total RNA per sample is required for Northern analysis of miRNA and their precursors, which is not feasible when the cell or tissue source is limited.

Alternatively, Allawi et al. (RNA 10:1153-1161, 2004) have developed a method for quantization of mature miRNAs using a modified Invader assay. Although apparently sensitive and specific for the mature miRNA, the drawback of the Invader quantization assay is the number of oligonucleotide probes and individual reaction steps needed for the complete assay, which increases the risk of cross-contamination between different assays and samples, especially when high-throughput analyses are desired.

Due to the small size of mature miRNAs, detecting them by standard RNA in situ hybridization has proven difficult to adapt in both plants and vertebrates, although in situ hybridization has recently been reported in A. thaliana and maize using RNA probes corresponding to the miRNA precursors (Chen et al., Science 203:2022-2025; 2004; Juarez et al., Nature 428:84-88; 2004). Brennecke et al. (Cell 113:25-36, 2003) and Mansfield et al. (Nature Genetics 36:1079-83, 2004) report on an alternative method in which reporter transgenes, so-called sensors, are designed and generated to detect the presence of a given miRNA in an embryo. Each sensor contains a constitutively expressed reporter gene (e.g., lacZ or green fluorescent protein) harboring miRNA target sites in its 3′-UTR. Thus, in cells that lack the miRNA in question, the transgene RNA is stable allowing detection of the reporter, whereas cells expressing the miRNA, the sensor mRNA is targeted for degradation by the RNAi pathway. Although sensitive, this approach is time-consuming since it requires generation of the expression constructs and transgenes. Furthermore, the sensor-based technique detects the spatiotemporal miRNA expression patterns via an indirect method as opposed to direct in situ hybridization of the mature miRNAs.

In contrast to low-throughput techniques previously employed, the large number of miRNAs and their precursors makes it difficult to create loss-of-function mutants for high-throughput genomic analyses. Another potential problem is that many miRNA genes are present in several copies per genome occurring in different loci, which makes it even more difficult to obtain mutant phenotypes. Boutla et al. (Nucleic Acids Research 31:4973-4980, 2003) describe the use of DNA antisense oligonucleotides complementary to 111 different miRNAs in Drosophila as well as their use to inactivate the miRNAs by injecting the DNA oligonucleotides into fly embryos. Of the 11 DNA antisense oligonucleotides, only 4 constructs showed severe interference with normal development, while the remaining 7 oligonucleotides didn't show any phenotypes presumably due to their inability to inhibit the miRNA in question. Thus, the success rate for using DNA antisense oligonucleotides to inhibit miRNA function would most likely be too low to allow functional analyses of miRNAs on a larger, genomic scale. An alternative approach to this has been reported by Hutvagner et al. (PLOS Biology 2:1-11, 2004), in which 2′-O-methyl antisense oligonucleotides could be used as potent and irreversible inhibitors of siRNA and miRNA function in vitro and in vivo in Drosophila and C. elegans, thereby inducing a loss-of-function phenotype. A drawback of this method is the need of high 2′-O-methyl oligonucleotide concentrations (100 micromolar) in transfection and injection experiments, which may be toxic to the animal.

High-Throughput Analysis of microRNAs and microRNA Precursors

DNA microarrays provide a high-throughput, genomic-scale alternative to other methods that quantify miRNAs and their precursors. Krichevsky et al. used cDNA microarrays to monitor the expression of miRNAs during neuronal development with 5 to 10 μg aliquot of input total RNA as target, but the mature miRNAs had to be separated from the miRNA precursors using micro concentrators prior to microarray hybridizations (Krichevsky et al., RNA 9:1274-1281, 2003). Liu et al. (Proc. Natl. Acad. Sci, U.S.A. 101:9740-9744, 2004) have developed a microarray for expression profiling of 245 human and mouse miRNAs using 40-mer DNA oligonucleotide probes. Thomson et al. (Nature Methods 1:1-6, 2004) describe the development of a custom oligonucleotide microarray platform for expression profiling of 124 mammalian miRNAs conserved in human and mouse using oligonucleotide probes complementary to the mature microRNAs. The microarray was used in expression profiling of the 124 miRNAs in question in different adult mouse tissues and embryonic stages. A similar approach was used by Miska et al. (Genome Biology 5:R68, 2004) for the development of an oligoarray for expression profiling of 138 mammalian miRNAs, including 68 miRNAs from rat and monkey brains. Yet another approach was taken by Barad et al. (Genome Research 14:2486-2494, 2004), who developed a 60-mer oligonucleotide microarray platform for known human mature miRNAs and their precursors.

The drawback of all DNA-based oligonucleotide arrays regardless of the oligonucleotide probe length is the requirement of high concentrations of labeled input target RNA for efficient hybridization and signal generation, low sensitivity for rare and low-abundant miRNA and their precursors, and the necessity for post-array validation using more sensitive assays such as real-time quantitative PCR, which is not currently feasible for mature miRNAs. In addition, at least in some array platforms discrimination of highly homologous miRNA differing by just one or two nucleotides could not be achieved, thus presenting problems in data interpretation, although the 60-mer microarray by Barad et al. (Genome Research 14:2486-2494, 2004) appears to have adequate specificity.

In conclusion, there is a need for new technologies that can detect, quantify, and functionally analyze miRNAs and miRNA precursors.

### SUMMARY OF THE INVENTION

The present invention improves the precision and sensitivity of the detection of miRNA precursors and their targets, compared to existing approaches. To this end, the invention provides oligonucleotide probes and a method for the design, synthesis, and use of oligonucleotide probes with improved sensitivity and high sequence specificity for miRNA precursors and their targets, e.g., mature miRNAs, mRNA, siRNAs, or other non-coding RNAs as well as miRNA precursor binding sites in their antisense RNAs or proteins that bind miRNA precursors. Such oligonucleotide probes include a sequence complementary to the desired RNA sequence and substitution with nucleotide analogues, preferably high-affinity nucleotide analogues, e.g., LNA, to increase their sensitivity and specificity over conventional oligonucleotides, such as DNA oligonucleotides, for hybridization to the desired RNA sequences.

An exemplary oligonucleotide probe includes a plurality of nucleotide analogue monomers and hybridizes to a miRNA precursor. Desirably, the nucleotide analogue is LNA, wherein the LNA may be oxy-LNA, preferably beta-D-oxy-LNA, monomers. Desirably, the oligonucleotide probe will hybridize to part of the loop sequence of a miRNA precursor, e.g., to 5 nucleotides of the miRNA precursor loop sequence or to the center of the miRNA precursor loop sequence. In other embodiments, the oligonucleotide probe will hybridize to part of the stem sequence of a miRNA precursor.

In other embodiments, two nucleotide analogue monomers are disposed 3 or 4 nucleotides apart, or a combination thereof. Alternatively, each nucleotide analogue monomer in a probe is spaced 3 or 4 nucleotides from the closest nucleotide analogue monomer. Typically, when nucleotide analogue monomers are spaced apart, only naturally-occurring nucleotides are disposed between the nucleotide analogue monomers. In another embodiment, two, three, four, or more nucleotide analogue monomers are disposed adjacent to one another. The adjacent nucleotide analogue monomers may be disposed at the 3′ or 5′ end or so that one of the nucleotide analogue monomers hybridizes to the center of the miRNA precursor. In other embodiments, the probe includes none or at most one mismatched base, deletion, or addition. Desirably, the probe hybridizes to the miRNA precursor under stringent conditions or high stringency conditions. In certain embodiments, the melting point of the duplex formed between the probe and the miRNA precursor is at least 1° C. higher, e.g., at least 5° C., than the melting point of the duplex formed between the miRNA precursor and a nucleic acid sequence not containing a nucleotide analogue monomer, e.g., LNA. The probe may include at least 70% DNA; at least 10% nucleotide analogue monomers; and/or at most 30% nucleotide analogue monomers. In addition, the probe may be at least 8 nucleotides long and at most 30 nucleotides long, e.g., 12 nucleotides long or 15 nucleotides long. The probe may further include a 5′ or 3′ amino group and/or a 5′ or 3′ label, e.g., a fluorescent (such as fluorescein) label, a radioactive label, or a label that is a complex having an enzyme (that may contain digoxigenin (DIG)). Other potential modifications of probes are described herein.

In other embodiments, the probe when hybridized to the miRNA precursor provides a substrate for RNase H; alternatively, the probe when hybridized to the miRNA precursor may not provide a substrate for RNase H. Preferably, the probes of the invention exhibit increases in binding affinity for the target sequence by at least two-fold compared to probes of the same sequence without the modification, under the same conditions for hybridization, e.g., stringent hybridization conditions.

The invention further features a method of creating a nucleotide duplex by providing a miRNA precursor; and contacting the miRNA precursor with a probe of the invention that hybridizes to the miRNA precursor; and the optional step of detecting the amount of a signal indicative of the miRNA precursor bound to the probe.

The invention also features a method of modulating, e.g., inhibiting or increasing, the biological activity of a miRNA precursor by providing the miRNA precursor; and contacting the miRNA precursor with a probe of the invention that hybridizes to the miRNA precursor, thereby modulating the biological activity of the miRNA precursor.

In addition, the invention features a method of determining the biological activity of a miRNA precursor by providing the miRNA precursor; contacting the miRNA precursor with a probe of the invention that hybridizes to the miRNA precursor; and assaying the biological activity.

The invention further features a method of comparing relative amounts of miRNA and miRNA precursor in a sample by contacting the sample with a first probe that hybridizes to the miRNA precursor and a second probe that hybridizes to the corresponding mature miRNA; and detecting the amount of one or more signals indicative of the relative amounts of miRNA and miRNA precursor.

The invention also features a method of measuring relative amounts of miRNA and miRNA precursor in a sample by contacting a first probe that hybridizes to mature miRNA with the sample under a first condition that also allows the corresponding miRNA precursor to hybridize; contacting the first probe or a second probe that hybridizes to mature miRNA with the sample under a second condition that does not allow corresponding miRNA precursor to hybridize; comparing the amounts of the probes hybridized under the two conditions wherein the reduction in amount hybridized under the second condition compared to the first condition is indicative of the amount of the miRNA precursor in the sample.

The invention also features methods of using the probes of the invention as components of Northern blots, in situ hybridization, arrays, bead-based arrays, and various forms of PCR analysis including PCR, RT-PCR, and qPCR.

Any method of the invention may involve probes containing labels. Any method of the invention may also involve contacting a probe with miRNA precursor that is endogenously or exogenously produced. Such contacting may occur in vitro or in vivo, e.g., such as in the body of an animal, or in a tissue sample, or within or without a cell, which may or may not naturally express the miRNA precursor.

Furthermore, the invention features a kit including a probe of the invention and packaging and/or labeling indicative of the miRNA precursor or possible targets of miRNA precursor, such as an miRNA, siRNA, other non-coding RNA, mRNA, RNA-edited transcript, or mRNA splice variants, to which the probe hybridizes and conditions under which the hybridization occurs. The kit provides for the isolation, purification, amplification, detection, identification, quantification, or capture of natural or synthetic nucleic acids. The probes are preferably immobilized onto a solid support, e.g., such as a bead or an array.

The invention also features a method of treating a disease or condition in a living organism using any combination of the probes and methods of the invention.

The probes of the invention and their corresponding methods have many advantages over existing technologies. The problems with existing detection, quantification and knock-down of miRNA precursors as outlined herein are addressed by the use of the oligonucleotide probes of the invention in combination with any of the methods of the invention selected so as to recognize or detect a majority of all discovered and detected miRNA precursors, in a given cell type from a given organism. By providing a sensitive and specific method for detection of miRNA precursors, e.g., such as mouse, rat, rabbit, monkey, or human miRNA precursors, the present invention overcomes the limitations discussed herein especially for conventional miRNA precursor assays.

Other features and advantages of the invention will be apparent from the following description, the figures, and the claims.

In the present context “ligand” means something that binds. Ligands include biotin, digoxigenin (DIG), and functional groups such as: aromatic groups (such as benzene, pyridine, naphthalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁-C₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-β-alanine, polyglycine, polylysine, peptides, oligo/polysaccharides, oligo/polyphosphates, toxins, antibiotics, cell poisons, and steroids, and also “affinity ligands”, i.e., functional groups or biomolecules that have a specific affinity for sites on particular proteins, antibodies, poly- and oligosaccharides, and other biomolecules.

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

“Transcriptome” refers to the complete collection of transcriptional units of the genome of any species. In addition to protein-coding mRNAs, it also represents non-coding RNAs, such as small nucleolar RNAs, siRNAs, microRNAs (miRNAs), microRNA precursors (pre-miRNAs), pri-miRNAs, and antisense RNAs, which have important structural and regulatory roles in the cell.

“Sample” refers to a sample of cells, or tissue or fluid isolated from an organism or organisms, including but not limited to skin, plasma, serum, spinal fluid, lymph fluid, synovial fluid, urine, tears, blood cells, organs, tumors, and also to samples of in vitro cell culture constituents (including but not limited to conditioned medium resulting from the growth of cells in cell culture medium, recombinant cells and cell components).

An “organism” refers to a living entity, including but not limited to human, mouse, rat, Drosophila, Caenorhabditis elegans (C. elegans), yeast, Arabidopsis thaliana, maize, rice, zebra fish, primates, domestic animals, etc.

The term “oligonucleotide probe” or “probe” refers to an oligonucleotide having a sequence (subsequently referred to as the “sequence of the oligonucleotide probe”) complementary to a RNA target sequence, and being substituted with high-affinity nucleotide analogues, e.g., LNA, to increase the sensitivity and specificity of conventional oligonucleotides, such as DNA oligonucleotides, for hybridization to short target sequences, e.g., miRNA precursors, mature miRNAs, pri-miRNAs, siRNAs or other non-coding RNAs as well as miRNA precursor binding sites in their cognate RNA targets, including other non-coding RNAs and mRNAs, mRNA splice variants, RNA-edited mRNAs and antisense RNAs.

The terms “miRNA” or “microRNA” or “mature miRNA” refer to 19-25 nt, e.g., 21-25, non-coding RNAs derived from endogenous genes. They are processed from longer (approximately 70 nucleotides in length) hairpin-like precursors termed pre-miRNAs. MicroRNAs assemble in complexes termed miRNPs and recognize their targets by antisense complementarity. If the microRNAs match 100% of their target, i.e., the complementarity is complete, the target mRNA is cleaved, and the miRNA acts like a siRNA. If the match is incomplete, i.e., the complementarity is partial, then the translation of the target mRNA is blocked.

The terms “microRNA precursor” or “miRNA precursor” or “pre-miRNA” refer to polynucleotide sequences (approximately 70 nucleotides in length) that form hairpin-like structures having a loop region and a stem region. The stem region includes a duplex created by the pairing of opposite ends of the pre-miRNA polynucleotide sequence. The loop region connects the two halves of the stem region. The pre-miRNAs are transcribed as mono- or poly-cistronic, long, primary precursor transcripts (pri-miRNAs) that are then cleaved into individual pre-miRNAs by a nuclear RNase III-like enzyme. Subsequently pre-miRNA hairpins are exported to the cytoplasm where they are processed by a second RNase III-like enzyme into miRNAs.

The “miRNA precursor loop sequence” or “loop sequence of the miRNA precursor” or “loop region” of an miRNA precursor is the portion of an miRNA precursor that is not present in the stem region and that is not retained in the mature miRNA (or its complement) upon cleavage by a RNAase III-like enzyme.

The “miRNA precursor stem sequence” or “stem sequence of the miRNA precursor” or “stem region” of an miRNA precursor is the portion of an miRNA precursor created by the pairing of opposite ends of the pre-miRNA polynucleotide sequence, and including the portion of the miRNA precursor that will be retained in the “mature miRNA.”

The “Hy3” and “Hy5” labels are commercially available dyes for labeling of proteins and modified oligonucleotides and are excited best by the 543 nm and 633 nm line, respectively, of a He—Ne laser.

The terms “small interfering RNA” or “siRNA” refer to 21-25 nt RNAs derived from processing of linear double-stranded RNA. siRNAs assemble in complexes termed RISC(RNA-induced silencing complex) and target homologous RNA sequences for endonucleolytic cleavage. Synthetic siRNAs also recruit RISCs and are capable of cleaving homologous RNA sequences

The term “RNA interference” (RNAi) refers to a phenomenon where double-stranded RNA homologous to a target mRNA leads to degradation of the targeted mRNA. More broadly defined as degradation of target mRNAs by homologous siRNAs.

The term “label” as used herein refers to any atom or molecule which can be used to provide a detectable (preferably quantifiable) signal, and which can be attached to a nucleic acid or protein. Labels may provide signals detectable by fluorescence, radioactivity, colorimetric, X-ray diffraction or absorption, magnetism, enzymatic activity, and the like or adhere to complexes that generate such signals. The label is detectable either by itself or as a part of a detection series. Examples of functional parts of labels are biotin, digoxigenin (DIG), fluorescent groups (groups which are able to absorb electromagnetic radiation, e.g., light or X-rays, of a certain wavelength, and which subsequently re-emits the energy absorbed as radiation of longer wavelength; illustrative examples are DANSYL (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radioisotopic labels, chemiluminescence labels (labels that are detectable via the emission of light during a chemical reaction), spin labels (a free radical, e.g., substituted organic nitroxides or other paramagnetic probes, e.g., Cu²⁺, Mg²⁺) bound to a biological molecule being detectable by the use of electron spin resonance spectroscopy). Preferred examples include biotin, fluorescein, Texas Red, rhodamine, dinitrophenyl, digoxigenin, Ruthenium, Europium, Cy5 and Cy3.

The term “complex comprising an enzyme” refers to any complex having an enzyme or having the properties of an enzyme, such as streptavidin or an antibody that binds digoxigenin (DIG) or an antibody that has a label, which may further include a secondary antibody that is fused to an enzyme which, for example, may facilitate staining, activate a reporter gene, modify a substrate, or generate some other signal.

The term “signal” refers to light, e.g., such as light generated by fluorescence, bioluminescence, or phosphorescence; radiation; particle emission; magnetism; staining; a product of a reaction involving an enzyme; or perturbations of a signal or signals, e.g., such as diffraction, absorbance, polarization, reflection, deflection, increases, decreases, or amplification of signals, that are indicative of an event, e.g., such as hybridization of a probe or probes to a polynucleotide or polynucleotides or binding of a protein to a probe.

As used herein, the terms “nucleic acid” and “polynucleotide” refer to primers, probes, oligomer fragments to be detected, oligomer controls and unlabeled blocking oligomers and are generic to polydeoxyribonucleotides (containing 2-deoxy-D-ribose), to polyribonucleotides (containing D-ribose), and to any other type of polynucleotide which is an N glycoside of a purine or pyrimidine base, or modified purine or pyrimidine bases. “Nucleic acid” and “polynucleotide” intend an oligonucleotide of genomic DNA or RNA, cDNA, semi-synthetic, or synthetic origin which, by virtue of its origin or manipulation: (1) is not associated with all or a portion of the polynucleotide with which it is associated in nature; and/or (2) is linked to a polynucleotide other than that to which it is linked in nature; and (3) is not found in nature. Because mononucleotides are reacted to make oligonucleotides in a manner such that the 5′-phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have a 5′ and 3′ ends. When two different, non-overlapping oligonucleotides anneal to different regions of the same linear complementary nucleic acid sequence, the 3′ end of one oligonucleotide points toward the 5′ end of the other; the former may be called the “upstream” oligonucleotide and the latter the “downstream” oligonucleotide. There is no intended distinction in length between the terms “nucleic acid” and “polynucleotide” and these terms will be used interchangeably. These terms refer only to the primary structure of the molecule. Thus, these terms include double- and single-stranded DNA, as well as double- and single stranded RNA. The oligonucleotide has a sequence of approximately at least 3 nucleotides, preferably at least about 6 nucleotides, and more preferably at least about 8-30 nucleotides corresponding to a region of the designated target nucleotide sequence. “Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

By the term “SBC nucleobases” is meant “Selective Binding Complementary” nucleobases, i.e., modified nucleobases that can make stable hydrogen bonds to their complementary nucleobases, but are unable to make stable hydrogen bonds to other SBC nucleobases. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T′ and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G′ and CG′. A stable hydrogen bonded pair is obtained when 2 or more hydrogen bonds are formed, e.g., the pair between A′ and T, A and T′, C′ and G′, and C′ and G. An unstable hydrogen bonded pair is obtained when 1 or no hydrogen bonds is formed, e.g., the pair between A′ and T′, and C′ and G′. Especially interesting SBC nucleobases are 2,6-diaminopurine (A′, also called D) together with 2-thio-uracil (U′, also called ^{2S}U)(2-thio-4-oxo-pyrimidine) and 2-thio-thymine (T′, also called ^{2S}T)(2-thio-4-oxo-5-methylpyrimidine).

“SBC LNA oligomer” refers to a “LNA oligomer” containing at least one LNA monomer where the nucleobase is a “SBC nucleobase”. By “LNA monomer with an SBC nucleobase” is meant a “SBC LNA monomer”. Generally speaking SBC LNA oligomers include oligomers that besides the SBC LNA monomer(s) contain other modified or naturally occurring nucleotides or nucleosides. By “SBC monomer” is meant a non-LNA monomer with a SBC nucleobase. By “isosequential oligonucleotide” is meant an oligonucleotide with the same sequence in a Watson-Crick sense as the corresponding oligonucleotide, e.g., the sequences agTtcATg is equal to agTscD^{2S}Ug where s is equal to the SBC DNA monomer 2-thio-t or 2-thio-u, D is equal to the SBC LNA monomer LNA-D and ^{2S}U is equal to the SBC LNA monomer LNA ^{2S}U.

The “complement” of a nucleic acid sequence or the “complementary” nucleic acid sequence as used herein refers to an oligonucleotide which, when aligned with the nucleic acid sequence such that the 5′ end of one sequence is paired with the 3′ end of the other, is in “antiparallel association.” Bases not commonly found in natural nucleic acids that may be included in the nucleic acids of the present invention include, for example, inosine and 7-deazaguanine. “Complementarity” may not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

When complementary nucleic acid sequences form a stable duplex, they are said to be “hybridized” or to “hybridize” to each other or it is said that “hybridization” has occurred.

Stability of a nucleic acid duplex is measured by the melting temperature, or “Tₘ”. The Tₘ of a particular nucleic acid duplex under specified conditions is the temperature at which half of the duplexes have disassociated.

The term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in U.S. Pat. No. 5,432,272 and Freier et al., Nucleic Acid Research, 25:4429-4443, 1997. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30:613-722, 1991, (see especially pages 622 and 623); in the J. I. Kroschwitz Ed., John Wiley & Sons, Concise Encyclopedia of Polymer Science and Engineering, 858-859, 1990; and in Cook, Anti-Cancer Drug Design, 6:585-607, 1991, each of which are hereby incorporated by reference in their entirety.

In addition to the aforementioned “nucleobases”, the term “nucleosidic base” or “nucleobase analogue” is further intended to include heterocyclic compounds that can serve as like nucleosidic bases including certain “universal bases” that are not nucleosidic bases in the most classical sense but serve as nucleosidic bases. Especially mentioned as a universal base is 3-nitropyrrole or a 5-nitroindole. Other preferred compounds include pyrene and pyridyloxazole derivatives, pyrenyl, pyrenylmethylglycerol derivatives and the like. Other preferred universal bases include, pyrrole, diazole or triazole derivatives, including those universal bases known in the art.

By “oligonucleotide,” “oligomer,” or “oligo” is meant a successive chain of monomers (e.g., glycosides of heterocyclic bases) connected via internucleoside linkages. The linkage between two successive monomers in the oligo consist of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═ (including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═ (including R⁵ when used as a linkage to a succeeding monomer), —CH₂—O—NR^{H}, —CO—NR^{H}—CH₂—, —CH₂—NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═ (including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —O—PO(OCH₂CH₃)—O—, —O—PO(OCH₂CH₂S—R^{H})—O—, —O—PO(BH₃)—O—, —O—PO(NHR^{N})—O—, —O—P(O)₂—NR^{H}—, —NR^{H}—P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially desirable. Further illustrative examples are given in Mesmaeker et al., Current Opinion in Structural Biology 5:343-355, 1995 and Susan M. Freier and Karl-Heinz Altmann, Nucleic Acids Research 25:4429-4443, 1997. The left-hand side of the internucleoside linkage is bound to the 5-membered ring as substituent P* at the 3′-position, whereas the right-hand side is bound to the 5′-position of a preceding monomer.

By “LNA” or “LNA monomer” (e.g., an LNA nucleoside or LNA nucleotide) or an LNA oligomer (e.g., an oligonucleotide or nucleic acid) is meant a nucleoside or nucleotide analogue that includes at least one LNA monomer. LNA monomers as disclosed in PCT Publication WO 99/14226 are in general particularly desirable modified nucleic acids for incorporation into an oligonucleotide of the invention. Additionally, the nucleic acids may be modified at either the 3′ and/or 5′ end by any type of modification known in the art. For example, either or both ends may be capped with a protecting group, attached to a flexible linking group, attached to a reactive group to aid in attachment to the substrate surface, etc. Desirable LNA monomers and their method of synthesis also are disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12:73-76, 2002; Hakansson et al., Bioorg. Med. Chem. Lett. 11:935-938, 2001; Koshkin et al., J. Org. Chem. 66:8504-8512, 2001; Kvaerno et al., J. Org. Chem. 66:5498-5503, 2001; Hakansson et al., J. Org. Chem. 65:5161-5166, 2000; Kvaerno et al., J. Org. Chem. 65:5167-5176, 2000; Pfundheller et al., Nucleosides Nucleotides 18:2017-2030, 1999; and Kumar et al., Bioorg. Med. Chem. Lett. 8(16):2219-2222, 1998.

Preferred LNA monomers, also referred to as “oxy-LNA” are LNA monomers which include bicyclic compounds as disclosed in PCT Publication WO 03/020739 wherein the bridge between R⁴* and R²* as shown in formula (I) below together designate —CH₂—O— or —CH₂—CH₂—O—.

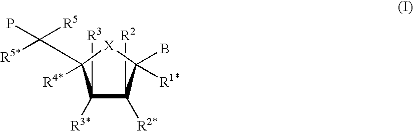

wherein X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*)—C(R⁷R⁷*).

B is selected from a modified base as discussed above, e.g., an optionally substituted carbocyclic aryl such as optionally substituted pyrene or optionally substituted pyrenylmethylglycerol, or an optionally substituted heteroalicylic or optionally substituted heteroaromatic such as optionally substituted pyridyloxazole, optionally substituted pyrrole, optionally substituted diazole or optionally substituted triazole moieties; hydrogen, hydroxy, optionally substituted C₁₋₄-alkoxy, optionally substituted C₁₋₄-alkyl, optionally substituted C₁₋₄-acyloxy, nucleobases, DNA intercalators, photochemically active groups, thermochemically active groups, chelating groups, reporter groups, and ligands.

P designates the radical position for an internucleoside linkage to a succeeding monomer, or a 5′-terminal group, such internucleoside linkage or 5′-terminal group optionally including the substituent R⁵. One of the substituents R², R²*, R³, and R³* is a group P* which designates an internucleoside linkage to a preceding monomer, or a 2′/3′-terminal group. The substituents of R¹*, R⁴*, R⁵, R⁵*, R⁶, R⁶*, R⁷, R⁷*, R^{N}, and the ones of R², R²*, R³, and R³* not designating P* each designates a biradical containing about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C=Q, wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₂-alkoxycarbonyl, C₁₋₁₂-alkylcarbonyl, formyl, aryl, aryloxy-carbonyl, aryloxy, arylcarbonyl, heteroaryl, hetero-aryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²*, R³, R³*, R⁴*, R⁵, R⁵*, R⁶ and R⁶*, R⁷, and R⁷* which are present and not involved in P, P* or the biradical(s) together may form an associated biradical selected from biradicals of the same kind as defined before; the pair(s) of non-geminal substituents thereby forming a mono- or bicyclic entity together with (i) the atoms to which the non-geminal substituents are bound and (ii) any intervening atoms.

Each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di-(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof.

Exemplary 5′, 3′, and/or 2′ terminal groups include —H, —OH, halo (e.g., chloro, fluoro, iodo, or bromo), optionally substituted aryl, (e.g., phenyl or benzyl), alkyl (e.g., methyl or ethyl), alkoxy (e.g., methoxy), acyl (e.g., acetyl or benzoyl), aroyl, aralkyl, hydroxy, hydroxyalkyl, alkoxy, aryloxy, aralkoxy, nitro, cyano, carboxy, alkoxycarbonyl, aryloxycarbonyl, aralkoxycarbonyl, acylamino, aroylamino, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, amidino, amino, carbamoyl, sulfamoyl, alkene, alkyne, protecting groups (e.g., silyl, 4,4′-dimethoxytrityl, monomethoxytrityl, or trityl(triphenylmethyl)), linkers (e.g., a linker containing an amine, ethylene glycol, quinone such as anthraquinone), detectable labels (e.g., radiolabels or fluorescent labels), digoxigenin (DIG), and biotin.

It is understood that references herein to a nucleic acid unit, nucleic acid residue, LNA monomer, or similar term are inclusive of both individual nucleoside units and nucleotide units and nucleoside units and nucleotide units within an oligonucleotide.

A “modified base” or other similar terms refer to a composition (e.g., a non-naturally occurring nucleobase or nucleosidic base), which can pair with a natural base (e.g., adenine, guanine, cytosine, uracil, and/or thymine) and/or can pair with a non-naturally occurring nucleobase or nucleosidic base. Desirably, the modified base provides a Tₘ differential of 15, 12, 10, 8, 6, 4, or 2° C. or less as described herein. Exemplary modified bases are described in EP 1 072 679 and WO 97/12896.

The term “chemical moiety” refers to a part of a molecule. “Modified by a chemical moiety” thus refer to a modification of the standard molecular structure by inclusion of an unusual chemical structure. The attachment of the structure can be covalent or noncovalent.

The term “inclusion of a chemical moiety” in an oligonucleotide probe thus refers to attachment of a molecular structure. Such chemical moieties include but are not limited to covalently and/or non-covalently bound minor groove binders (MGB) and/or intercalating nucleic acids (INA) selected from a group consisting of asymmetric cyanine dyes, DAPI, SYBR Green I, SYBR Green II, SYBR Gold, PicoGreen, thiazole orange, Hoechst 33342, Ethidium Bromide, 1-O-(1-pyrenylmethyl)glycerol and Hoechst 33258. Other chemical moieties include the modified nucleobases, nucleosidic bases, or LNA.

“Oligonucleotide analogue” refers to a nucleic acid binding molecule capable of recognizing a particular target nucleotide sequence. A particular oligonucleotide analogue is peptide nucleic acid (PNA) in which the sugar phosphate backbone of an oligonucleotide is replaced by a protein-like backbone. In PNA, nucleobases are attached to the uncharged polyamide backbone yielding a chimeric pseudopeptide-nucleic acid structure, which is homomorphous to nucleic acid forms.

A “nucleotide analogue” or “nucleotide analogue monomer” is any compound structurally similar or functionally similar to a nucleotide. Nucleotide analogues may include nucleotides that contain or nucleotides that are, for example, high affinity nucleotide analogues, LNAs, nucleobases, nucleobase analogues, modified bases, SBC nucleobases, SBC monomers, and modified backbones.

“High affinity nucleotide analogue” refers to a non-naturally occurring “nucleotide analogue” that increases the “binding affinity” of an oligonucleotide probe to its complementary sequence when substituted with at least one such high-affinity nucleotide analogue.

As used herein, a probe with an increased “binding affinity” for a sequence compared to a probe which has the same sequence but does not have a stabilizing nucleotide, refers to a probe for which the association constant (Kₐ) of the probe recognition segment is higher than the association constant of the complementary strands of a double-stranded molecule. In another preferred embodiment, the association constant of the probe recognition segment is higher than the dissociation constant (K_{d}) of the complementary strand of the sequence in the target sequence in a double stranded molecule.

Monomers are referred to as being “complementary” if they contain nucleobases that can form hydrogen bonds according to Watson-Crick base-pairing rules (e.g., G with C, A with T or A with U) or other hydrogen bonding motifs such as for example diaminopurine with T, 5-methyl C with G, 2-thiothymidine with A, inosine with C, pseudoisocytosine with G, etc.

The term “succeeding monomer” relates to the neighboring monomer in the 5′-terminal direction and the “preceding monomer” relates to the neighboring monomer in the 3′-terminal direction.

The term “target nucleic acid” or “target ribonucleic acid” refers to any relevant nucleic acid of a single specific sequence, e.g., a biological nucleic acid, e.g., derived from a patient, an animal (a human or non-human animal), a plant, a bacteria, a fungi, an archae, a cell, a tissue, an organism, etc. For example, where the target ribonucleic acid or nucleic acid is derived from a bacteria, archae, plant, non-human animal, cell, fungi, or non-human organism, the method optionally includes selecting the bacteria, archae, plant, non-human animal, cell, fungi, or non-human organism based upon detection of the target nucleic acid. In one embodiment, the target nucleic acid is derived from a patient, e.g., a human patient. In this embodiment, the invention optionally further includes selecting a treatment, diagnosing a disease, or diagnosing a genetic predisposition to a disease, based upon detection of the target nucleic acid.

“Target sequence” refers to a specific nucleic acid sequence within any target nucleic acid.

The term “stringent conditions”, as used herein, is the “stringency” which occurs within a range from about Tₘ−5° C. (5° C. below the melting temperature (Tₘ) of the probe) to about 20° C. to 25° C. below Tₘ. As will be understood by those skilled in the art, the stringency of hybridization may be altered in order to identify or detect identical or related polynucleotide sequences. Hybridization techniques are generally described in Nucleic Acid Hybridization, A Practical Approach, Ed. Hames, B. D. and Higgins, S. J., IRL Press, 1985; Gall and Pardue, Proc. Natl. Acad. Sci., USA 63:378-383, 1969; and John, et al. Nature 223:582-587, 1969, each of which is hereby incorporated by reference. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Hybridization under “low stringency” can be obtained in the absence of organic solvent, e.g., formamide, while hybridization under “high stringency” or “high stringency conditions” can be obtained in the presence of at least about 35% formamide, and most preferably at least about 50% formamide and optionally at temperatures higher than stringent or low stringency conditions. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Additional variations on these conditions will be readily apparent to those skilled in the art. Hybridization techniques are well known to those skilled in the art and are described, for example, in Benton and Davis, Science 196:180, 1977; Grunstein and Hogness, Proc. Natl. Acad. Sci., USA 72:3961, 1975; Ausubel et al., Current Protocols in Molecular Biology, Wiley Interscience, New York, 2001; Berger and Kimmel, Guide to Molecular Cloning Techniques, 1987, Academic Press, New York; and Sambrook et al., Molecular Cloning: A Laboratory Manual, Cold Spring Harbor Laboratory Press, New York.

By “modified backbone” is meant a nucleotide backbone structure other than the naturally occurring ribose-phosphate or deoxyribose-phosphate backbones. Exemplary modified backbones include a ribonucleotide moiety that is substituted at the 2′ position. The substituents at the 2′ position may, for example, be a saturated, unsaturated, unbranched, or branched C1 to C4 alkyl group, e.g., 2′-O-methyl ribose. Another suitable example of a substituent at the 2′ position of a modified ribonucleotide moiety is a C1 to C4 alkoxy-C1 to C4 alkyl group, e.g., methoxyethyl. Another suitable example of a modified ribonucleotide moiety is a ribonucleotide that is substituted at the 2′ position with fluoro group. Preferred modified backbones also include LNA.

By two nucleotides “disposed X nucleotides apart” is meant positioned in a nucleotide sequence so that X-1 nucleotides are disposed between the two nucleotides. For example, in the sequence ACTG, the A and G are disposed three nucleotides apart.

### BRIEF DESCRIPTION OF DRAWINGS

The patent or application file contains at least one drawing executed in color. Copies of this patent or patent application publication with color drawings will be provided by the Office upon request and payment of the necessary fee.

FIG. 1. Plot of Hy3-labeled total human brain RNA before fractionation (x-axis) versus Hy5-labeled flashPAGE human brain RNA after fractionation (y-axis). Probes designed to hybridize shorter mature miRNAs have a decreased signal after fractionation, which removes longer transcripts, suggesting that longer transcripts, such as miRNA precursors, are also binding the mature miRNA probes. The signal has been background subtracted and the total intensity from each channel has been normalized.

FIG. 2. Hy3 channel measurements with and without miRNA precursor spike-in, showing the signal from each oligonucleotide probe (an average of 4 identical replicas on each slide). The spike-in of miRNA precursor elevates signal on both mature miRNA and miRNA precursor probes. The signal has been background subtracted and the total intensity from each channel has been normalized. The scale is logarithmic.

FIG. 3. Northern blot analysis of different brain specific miRNAs. 30 μg of total RNA from HeLa cells and from different mouse tissues were blotted and probed with a 5′-radiolabeled, LNA-containing oligodeoxynucleotide complementary to the indicated miRNAs. Equal loading of total RNA on the gel was verified by probing with a probe complementary to the U6 snRNA. Note that only miR-138 displays a ubiquitously transcribed precursor.

FIG. 4. Northern blot analysis of total RNA isolated from HeLa and N2A cells and from different murine tissues showing the tissue-specific processing of the ubiquitous 69-nt pre-miR-138-2. The band at 69-nt corresponds to the precursor, which is present in all cells and tissues that were analyzed. The 23-24 nt bands represent the mature miR-138, which is specifically expressed in the cerebrum as well as in the cerebellum and N2A cells, albeit to a lower extent.

FIG. 5A. In situ hybridization experiment on cryo-sections of E17 mouse embryos using oligonucleotide probes containing LNA that recognize mature miR-138 (left panel) or pre-miR-138-2 (right panel). The probe against the mature miRNA shows a strong expression in neuronal tissues (brain, CNS) and also in the fetal liver, while the probe hybridizing to the precursor gives a strong signal in almost all tissues of the embryo.

FIG. 5B. In situ hybridization experiments on cryo-sections of adult mouse brain using oligonucleotide probes containing LNA that recognize mature miR-138 (left panel) or pre-miR-138-2 (right panel). miR-138 is primarily located to specific regions of the neocortex, most neurons in the hippocampus and granule and purkinje cells of the cerebellum, while premiR-138-2 is essentially uniformly distributed.

FIG. 6A. High magnification images of in situ hybridization experiments on cryosections of adult mouse cerebellum, showing the expression of mature miR-138 in the granule cells (G) and in the Purkinje cell layer (P) of the cerebellum.

FIG. 6B. The levels of pre-miR-138-2 are lower in the granule cells (G) and in the Purkinje cell layer (P) of the cerebellum, indicating an efficient conversion of pre-miR-138-2 into the mature miR-138, and a consequent depletion of the precursor.

FIG. 7. Northern blot of total HeLa RNA and cytoplasmic and nuclear RNA fractions from HeLa cells probed against pre-miR-138-2. The band at 69-nt corresponds to premiR-138-2, that is mainly found in the cytoplasmic fraction, indicating nuclear export of the precursor.

FIG. 8A. Processing reaction of pre-miR-138-2 into mature miR-138 by recombinant Dicer (rDcr) resolved on a 15% denaturing PAGE. The band corresponding to the respective pre-miRNA is indicated by ‘pre’, whereas the band representing the mature miRNA is designated by ‘m’.

FIG. 8B. The addition of increasing amounts of HeLa cytoplasmic extract specifically abolishes the processing of pre-miR-138-2. The top band corresponds to the respective pre-miRNA, whereas the bottom band corresponds to the mature miRNA.

FIG. 8C. Processing of pre-miR-19a into mature miR-19a remains unaffected by the addition of high amounts of HeLa cytoplasmic extract. The band corresponding to the respective pre-miRNA is indicated by ‘pre’, whereas the band representing the mature miRNA is designated by ‘m’.

FIG. 9. Model depicting how differential processing of an otherwise ubiquitously expressed pre-miRNA mediates tissue- and/or developmental stage-specific expression of the mature miRNA. In both tissues, expressing or not expressing the mature miR-138, its gene is transcribed into pri-miR-138-2, which is cleaved in the nucleus by Drosha into pre-miR-138-2, and then exported to the cytoplasm. Here, the presence or absence of an inhibitory factor determines, if pre-miR-138-2 becomes processed or not, thus leading to differential expression of mature miR-138.

FIG. 10A. Multiple sequence alignments of the flanking regions of both chromosomal loci encoding miR-138. Regions in red represent >90%, and regions in green 50-90% sequence homology. Note, that the pre-miR-138-2 surrounding sequences are highly conserved, while this is not the case for miR-138-1. (Abbreviations: Mm=Mus musculus; Rⁿ=Rattus norvegicus; Cf=Canis familiaris).

FIG. 10B. Northern blot of pre-miR-138-1 and pre-miR-138-2. 100 μg of total RNA from murine brain were blotted and probed with a 5′-radiolabeled, oligonucleotide probes containing LNA complementary to the mature sequence of miR-138 (m), to the terminal loop of pre-miR-138-1 (p1) and to the terminal loop of pre-miR-138-2 (p2).

FIG. 11. A 37-plex of microspheres coupled with oligonucleotide probes, three of which are control oligonucleotide probes, were used for the analyses of total RNA sample from human colon. The results are shown. Please note that the MFI reading for the negative control, no template control is around 45 (data not shown).

FIG. 12. Fold difference generated from addition of pre-hsa-miR-10a in a yeast RNA background when comparing the signal to a yeast RNA only background. Only very little increase in signal was observed from the hsa-miR-10b capture probe, which only has a single mismatch internally compared to hsa-miR-10a. The signal is the average of two flip dye experiments with four replicates on each slide.

FIG. 13. Fold difference generated from addition of pre-hsa-miR-15a in a yeast RNA background when comparing the signal to a yeast RNA only background. No significant signal was observed from the hsa-miR-15b capture probe that only has a single mismatch internally compared to hsa-miR-15a. The signal is the average of two flip dye experiments with four replicates on each slide.

FIG. 14. Fold difference generated from addition of pre-hsa-miR-26a-1 in a yeast RNA background when comparing the signal to a yeast RNA only background. Some increase in signal was observed from the hsa-miR-26b capture probe which has two mismatches, one internally and one in the end, compared to hsa-miR-26a. The high signal from hsa-miR-26a, indicates that a significant amount of truncated synthetic miRNA exists. No change in signal was observed from either pre-hsa-miR-26a-2 or pre-hsa-miR-26b. The signal is the average of two flip dye experiments with four replicates on each slide.

FIG. 15. Fold difference generated from addition of pre-hsa-miR-99a in a yeast RNA background when comparing the signal to a yeast RNA only background. No significant signal was observed from the pre-hsa-miR-99b capture probe, but some cross hybridization was observed at hsa-miR-99b, which has triple internal mismatches compared to hsa-miR-99a. The signal is the average of two flip dye experiments with four replicates on each slide.

### DETAILED DESCRIPTION OF THE INVENTION

The present invention improves the precision and sensitivity of the detection of miRNA precursors and their targets. To this end, the invention provides oligonucleotide probes and methods for the design, synthesis, and use of the oligonucleotide probes with improved sensitivity and high sequence specificity for miRNA precursors and their targets, e.g., mature miRNAs, mRNA, siRNAs, or other non-coding RNAs as well as miRNA precursor binding sites in their antisense RNAs or proteins that bind miRNA precursors. In general, oligonucleotide probes of the invention include a sequence complementary to the desired RNA sequence, wherein the sequence is substituted with high-affinity nucleotide analogues, e.g., LNA, to increase the sensitivity and specificity over conventional oligonucleotides, such as DNA oligonucleotides, for hybridization to the desired RNA sequences.

### Oligonucleotide Probes

Typically, an oligonucleotide probe of the invention includes a plurality of nucleotide analogue monomers and hybridizes to a miRNA precursor. Desirably, the nucleotide analogue is LNA, wherein the LNA are oxy-LNA, preferably beta-D-oxy-LNA, monomers. Desirably, the oligonucleotide probe hybridizes to the loop sequence of a miRNA precursor, e.g., to the majority of the nucleotides of the miRNA precursor loop sequence or to the center of the miRNA precursor loop sequence. The oligonucleotide probe may or may not also hybridize to the stem sequence of the miRNA precursor. The oligonucleotide probe may have a number of nucleotide analogue monomers corresponding to 20% to 40% of the probe oligonucleotides. The probes may also have a spacing between nucleotide analogue monomers such that two of the plurality of nucleotide analogue monomers are disposed 3 or 4 nucleotides apart, or a combination thereof. Alternatively, each nucleotide analogue monomer in a probe may be spaced 3 or 4 nucleotides from the closest nucleotide analogue monomer. Typically, when nucleotide analogue monomers are spaced apart, only naturally-occurring nucleotides are disposed between the nucleotide analogue monomers. Alternatively, two, three, four, or more nucleotide analogue monomers may be disposed adjacent to one another. The adjacent nucleotide analogue monomers may or may not be disposed at the 3′ or 5′ end of the oligonucleotide probe or so that one of the nucleotide analogue monomers hybridizes to the center of the loop sequence of the miRNA precursor. The probe may include none or at most one mismatched base, deletion, or addition. Desirably, the probe hybridizes to the miRNA precursor under stringent conditions or high stringency conditions. Desirably, the melting point of the duplex formed between the probe and the miRNA precursor is at least 1° C. higher, e.g., at least 5° C., than the melting point of the duplex formed between the miRNA precursor and a nucleic acid sequence not having a nucleotide analogue monomer, or any modified backbone. The probe may include at least 70% DNA; at least 10% nucleotide analogue monomers; and/or at most 30% nucleotide analogue monomers.

Desirably, the oligonucleotide probes may be modified using the chimeric or ‘gapmer’ approach, which entails having stable modified nucleotides at the 5′ and 3′ ends of the probe with nucleotides having phosphodiester or phosphorothioate bonds in the center of the probe to enhance probe stability in a cellular environment. (Kang et al., Nucleic Acids Research 32:4411-4419, 2004; U.S. 2005/0203042). Other modifications to the probes that also mitigate non-specific probe binding to certain cell proteins, lack of probe activation by RNase H, or poor cellular uptake of the probe may also be made. Similar modifications can be made to the probes using the ribozyme approach for therapeutic purposes.

The probe may further include a 5′ or 3′ amino group and/or a 5′ or 3′ label, e.g., a fluorescent (such as fluorescein) label, a radioactive label, or a label that is a complex including an enzyme (such as a complex containing digoxigenin (DIG)). The probe is for example 8 nucleotides to 30 nucleotides long, e.g., 12 nucleotides long or 15 nucleotides long. Other potential modifications of probes are described herein.

The probe when hybridized to the miRNA precursor may or may not provide a substrate for RNase H. Preferably, the probes of the invention exhibit increased binding affinity for the target sequence by at least two-fold, e.g., at least 5-fold or 10-fold, compared to probes of the same sequence without nucleotide analogue monomers, under the same conditions for hybridization, e.g., stringent conditions or high stringency conditions.

The invention also features a second oligonucleotide probe which includes a plurality of nucleotide analogue monomers and hybridizes to a mature miRNA. Desirably, the nucleotide analogue is LNA, wherein the LNA are oxy-LNA, preferably beta-D-oxy-LNA, monomers. Desirably, the second oligonucleotide probe will hybridize to part of the mature miRNA. The second oligonucleotide probe may have a number of nucleotide analogue monomers corresponding to 20% to 40% of the second probe's oligonucleotides. The second probe may also have a spacing between nucleotide analogue monomers such that two of the plurality of nucleotide analogue monomers are disposed 3 or 4 nucleotides apart, or a combination thereof. Alternatively, each nucleotide analogue monomer in the second probe may be spaced 3 or 4 nucleotides from the closest nucleotide analogue monomer. Typically, when nucleotide analogue monomers are spaced apart, only naturally-occurring nucleotides are disposed between the nucleotide analogue monomers. Alternatively, two, three, four, or more nucleotide analogue monomers may be disposed adjacent to one another. The adjacent nucleotide analogue monomers may or may not be disposed at the 3′ or 5′ end of the second oligonucleotide probe. The second probe may include none or at most one mismatched base, deletion, or addition. Desirably, the second probe hybridizes to the mature miRNA under stringent conditions or high stringency conditions. Desirably, the melting point of the duplex formed between the second probe and the mature miRNA is at least 1° C. higher, e.g., at least 5° C., than the melting point of the duplex formed between the mature miRNA and a nucleic acid sequence not having a nucleotide analogue monomer, or any modified backbone. The second probe may include at least 70% DNA; at least 10% nucleotide analogue monomers; and/or at most 30% nucleotide analogue monomers.

### Software

The invention features a computer code for a preferred software program of the invention for the design and selection of the oligonucleotide probes. The invention provides a method, system, and computer program embedded in a computer readable medium (“a computer program product”) for designing oligonucleotide probes having at least one stabilizing nucleobase, e.g., such as LNA. The method includes querying a database of target sequences (e.g., such as the miRNA registry at http://www.sanger.ac.uk/Software/Rfam/mirna/index.shtml) and designing oligonucleotide probes which: i) have sufficient binding stability to bind their respective target sequence under stringent hybridization conditions, ii) have limited propensity to self-anneal (i.e., to hybridize to an identical oligonucleotide), and iii) are capable of binding to and detecting/quantifying at least about 60%, at least about 70%, at least about 80%, at least about 90% or at least about 95% of their target sequences in the given database of miRNAs, miRNA precursors, or other RNA sequences.

The target sequence database may include nucleic acid sequences corresponding to human, mouse, rat, Drosophila melanogaster, C. elegans, Arabidopsis thaliana, maize, or rice miRNAs.

The method further entails calculating stability based on the assumption that the oligonucleotide probes have at least one stabilizing nucleotide, such as an LNA monomer. In some cases, the calculated stability is used to eliminate oligonucleotide probe candidates with inadequate stability from the database of possible oligonucleotide probes prior to the query against the database to identify optimal sequences for the oligonucleotide probe.

### Synthesis

Methods of designing an oligonucleotide probe by selecting optimal substitution patterns for the high-affinity analogues, e.g., LNAs or other nucleotide analogues, in the sequence of the oligonucleotide probe, include the oligonucleotide probes with nucleotide analogue monomers using regular spacing between the nucleotide analogue monomers, e.g., at every second nucleotide position, every third nucleotide position, or every fourth nucleotide position, in order to promote the A-type duplex geometry between the oligonucleotide probe and its complementary RNA target, with each possible combination of nucleotide analogue monomer substitution present in at least one of the probes designed and with an unmodified monomer at the 5′-end position and 3′-end position; determining the ability of the designed oligonucleotide probes to self-anneal; determining the melting temperature of the substituted oligonucleotide probes of the invention hybridized to a complementary sequence; and selecting the oligonucleotide probes with the highest melting temperatures and least ability to self-anneal. The binding affinity of the selected oligonucleotide probe containing nucleotide analogue is desirably at least about 3-fold, 4-fold, 5-fold, or 20-fold higher than the binding of a probe of the same sequence but without nucleotide analogue or any other stabilizing feature.

Furthermore, a method of designing the oligonucleotide probes by selecting optimal substitution patterns for the nucleotide analogues includes substituting the sequence of the oligonucleotide probe with nucleotide analogues using irregular spacing between the nucleotide analogue monomers and selecting the oligonucleotide probes with the highest melting temperatures and least ability to self-anneal, as described above.

Once the appropriate RNA sequences have been selected, nucleotide analogue substituted oligonucleotide probes, e.g., LNA-substituted probes, are preferably chemically synthesized using commercially available methods and equipment as described in the art (Koshkin et al., Tetrahedron 54:3607-30, 1998). For example, the solid phase phosphoramidite method can be used to produce short LNA probes (Caruthers, et al., Cold Spring Harbor Symp. Quant. Biol. 47:411-418, 1982; Adams, et al., J. Am. Chem. Soc. 105:661-663, 1983). Desirable LNA monomers and their method of synthesis are also disclosed in U.S. Pat. No. 6,043,060, U.S. Pat. No. 6,268,490, PCT Publications WO 01/07455, WO 01/00641, WO 98/39352, WO 00/56746, WO 00/56748 and WO 00/66604 as well as in the following papers: Morita et al., Bioorg. Med. Chem. Lett. 12:73-76, 2002; Hakansson et al., Bioorg. Med. Chem. Lett. 11:935-938, 2001; Koshkin et al., J. Org. Chem. 66:8504-8512, 2001; Kvaerno et al., J. Org. Chem. 66:5498-5503, 2001; Hakansson et al., J. Org. Chem. 65:5161-5166, 2000; Kvaerno et al., J. Org. Chem. 65:5167-5176, 2000; Pfundheller et al., Nucleosides Nucleotides 18:2017-2030, 1999; Kumar et al., Bioorg. Med. Chem. Lett. 8:2219-2222, 1998.

Probe sequences may also include Selectively Binding Complementary (SBC) nucleobases, i.e., modified nucleobases that can make stable hydrogen bonds to their complementary nucleobases, but are unable to make stable hydrogen bonds to other SBC nucleobases. Such SBC nucleobase substitutions, which may be LNA or non-LNA, are especially useful when highly self-complementary oligonucleotide probes are employed. As an example, the SBC nucleobase A′, can make a stable hydrogen bonded pair with its complementary unmodified nucleobase, T. Likewise, the SBC nucleobase T′ can make a stable hydrogen bonded pair with its complementary unmodified nucleobase, A. However, the SBC nucleobases A′ and T′ will form an unstable hydrogen bonded pair as compared to the base pairs A′-T′ and A-T′. Likewise, a SBC nucleobase of C is designated C′ and can make a stable hydrogen bonded pair with its complementary unmodified nucleobase G, and a SBC nucleobase of G is designated G′ and can make a stable hydrogen bonded pair with its complementary unmodified nucleobase C, yet C′ and G′ will form an unstable hydrogen bonded pair as compared to the base pairs C′-G′ and CG′. A stable hydrogen bonded pair is obtained when 2 or more hydrogen bonds are formed, e.g., the pair between A′ and T, A and T′, C′ and G′, and C′ and G. An unstable hydrogen bonded pair is obtained when 1 or no hydrogen bonds is formed, e.g., the pair between A′ and T′, and C′ and G′. Especially interesting SBC nucleobases are 2,6-diaminopurine (A′, also called D), together with 2-thio-uracil (U′, also called 2SU)(2-thio-4-oxo-pyrimidine), and 2-thio-thymine (T′, also called 2ST)(2-thio-4-oxo-5-methylpyrimidine). Other SBCs have decreased ability to self-anneal or to form duplexes with oligonucleotide probes containing one or more modified bases.

Probes of the invention may further include any number of modifications. Probes may be labeled or conjugated to other functional moieties, e.g., through the attachment of duplex-stabilizing agents such as minor-groove-binders (MGB) or intercalating nucleic acids (INA). Additionally, the modifications may also include addition of non-discriminatory bases, e.g., such as 5-nitroindole, which are capable of stabilizing duplex formation regardless of the nucleobase at the opposing position on the target strand. The modifications may also include the addition of naturally and non-naturally occurring nucleobases including those disclosed in U.S. Pat. No. 3,687,808; in chapter 15 by Sanghvi, in Antisense Research and Application, Ed. S. T. Crooke and B. Lebleu, CRC Press, 1993; in Englisch, et al., Angewandte Chemie, International Edition, 30:613-722, 1991, (see especially pages 622 and 623); in the J. I. Kroschwitz Ed., John Wiley & Sons, Concise Encyclopedia of Polymer Science and Engineering, 858-859, 1990; and in Cook, Anti-Cancer Drug Design, 6:585-607, 1991, each of which are hereby incorporated by reference in their entirety. Exemplary modified bases are also described in EP 1 072 679 and WO 97/12896.

Oligonucleotide probes may also include a ligand, e.g., such as a drug or a ligand that can be bound by an antibody. Such ligand-containing oligonucleotide probes of the invention are useful for isolating target RNA molecules from complex nucleic acid mixtures, such as miRNA precursors and their cognate target RNAs or proteins. Exemplary ligands include biotin, digoxigenin (DIG), and functional groups such as: aromatic groups (e.g., benzene, pyridine, naphthalene, anthracene, and phenanthrene), heteroaromatic groups (e.g., thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicar-bazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C1-C20 alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-α-alanine, polyglycine, polylysine, peptides, oligo/polysaccharides, oligo/polyphosphates, toxins, antibiotics, cell poisons, and steroids, and also affinity ligands, i.e., functional groups or biomolecules that have a specific affinity for sites on particular proteins, antibodies, poly- and oligosaccharides, and other biomolecules. One or more ligands that increase cell membrane permeability, e.g., cell penetration enhancers, may also be included in the probes. Exemplary ligands that increase cell membrane permeability include lipophilic groups (e.g., sterols such as cholesterols, lanosterol, phytosterols, adamantols, fatty acids), peptides (e.g., cell penetrating peptides), charged moieties, functionalized alkyls, functionalized heteroalkyls, or other moieties assisting cellular uptake of oligonucleotides passively or actively.

Oligonucleotide probes according to the invention may include single labels or a plurality of labels, e.g., a pair of labels that interact with each other either to produce a signal or to produce a change in a signal when hybridization of the oligonucleotide probe to a target sequence occurs. The oligonucleotide probe may include a fluorophore moiety and a quencher moiety, positioned in such a way that the hybridized state of the probe can be distinguished from the unhybridized state of the probe by an increase in the fluorescent signal from the oligonucleotide probe. The oligonucleotide probe may have, in addition to a sequence that hybridizes to a sequence in a target molecule, first and second complementary sequences which specifically hybridize to each other when the probe is not hybridized to a sequence in a target molecule, bringing the quencher molecule in sufficient proximity to the reporter molecule to quench fluorescence of the reporter molecule. Hybridization of the target molecule distances the quencher from the fluorophores moiety and results in a signal proportional to the amount of hybridization.

Many synthetic approaches can be employed to add the above moieties, ligands, and labels to nucleotide analogue-containing-probes. The flexibility of the phosphoramidite synthesis approach furthermore facilitates the easy production of LNAs carrying all commercially available linkers, fluorophores and labeling-molecules available for this standard chemistry. Oligonucleotide probes containing LNA may also be labeled by enzymatic reactions, e.g., by kinasing using T4 polynucleotide kinase and gamma-³²P-ATP or by using terminal deoxynucleotidyl transferase (TDT) and any given digoxigenin-conjugated nucleotide triphosphate (dNTP) or dideoxynucleotide triphosphate (ddNTP).

The oligonucleotide probes of the invention may be covalently bonded to a solid support, e.g., by reaction of a nucleoside phosphoramidite with an activated solid support, and subsequent reaction of a nucleoside phosphoramide with an activated nucleotide or nucleic acid bound to the solid support. Preferably, the solid support or the oligonucleotide probes bound to the solid support is activated by illumination, a photogenerated acid, or electric current. Alternatively, the oligonucleotide probes may contain a spacer, e.g., a randomized nucleotide sequence or a non-base sequence, such as hexaethylene glycol, between the reactive group and the rest of the oligonucleotide probe. Such covalently bonded oligonucleotide probes are highly useful for large-scale detection and expression profiling of miRNA precursors, the targets of miRNA precursors, and other coding or non-coding RNAs. The oligonucleotide probes may also have a photochemically active group, a thermochemically active group, a chelating group, a reporter group, or a ligand that facilitates the direct or indirect detection of the probe or the immobilization of the oligonucleotide probe onto a solid support. Alternatively, the photochemically active group, the thermochemically active group, the chelating group, the reporter group, or the ligand includes a spacer (K) having a chemically cleavable group; or the photochemically active group, the thermochemically active group, the chelating group, the reporter group, or the ligand may be attached via the biradical of at least one of the nucleotide analogues of the oligonucleotide.

The solid support may contain a material, e.g., selected from borosilicate glass, soda-lime glass, polystyrene, polycarbonate, polypropylene, polyethylene, polyethyleneglycol terephthalate, polyvinylacetate, polyvinylpyrrolidinone, polymethylmethacrylate and polyvinylchloride, preferably polystyrene and polycarbonate. The solid support may be in the form of a specimen tube, a vial, a slide, a sheet, a film, a bead, a pellet, a disc, a plate, a ring, a rod, a net, a filter, a tray, a microtitre plate, a stick, or a multi-bladed stick.

A probe of the invention may be assembled in a kit with packaging and/or labeling indicative of the miRNA precursor or possible target of miRNA precursor, such as an miRNA, siRNA, other non-coding RNA, mRNA, RNA-edited transcript, or mRNA splice variants, to which the probe hybridizes and conditions under which the hybridization occurs. The may be used for the isolation, purification, amplification, detection, identification, quantification, or capture of natural or synthetic nucleic acids. The probes may be immobilized onto a solid support or derivatized for attachment by the end user.

The invention also features probes, as described herein, in combination with a pharmaceutically acceptable carrier. Such carriers are known in the art.

Exemplary miRNA precursors are described herein and are known in the art, e.g., in U.S. 2004/0053411; U.S. 2004/0175732; U.S. 2004/0268441; U.S. 2005/0059005; U.S. 2005/0075492; U.S. 2005/0144669; U.S. 2005/0260648; U.S. 2005/0261218; U.S. 2005/0266418; U.S. 2005/0266552; U.S. 2005/0277139; U.S. 2006/0019286; U.S. 2006/0057595; U.S. 2006/0058266; U.S. 2006/0063174; U.S. 2006/0078894; and U.S. 2006/0088864.

Exemplary miRNA are also described herein and are known in the art, e.g., in U.S. 2005/0182005; WO 2005/013901, and the miRBase Sequence Database (Nucleic Acids Research: Database issue, 34:D140-D144, 2006), each of which is hereby incorporated by reference.

Other methods of synthesizing and labeling oligonucleotide probes or monomers are known in the art.

### Applications

The present invention provides oligonucleotide probes for the use in detection, isolation, purification, amplification, identification, quantification, or capture of miRNA precursors and their targets, potentially including miRNAs, siRNAs, other non-coding RNAs, mRNAs, RNA-edited transcripts or alternative mRNA splice variants.

The oligonucleotide probes of invention are useful for the capture and detection of individual RNA molecules in complex mixtures composed of hundreds of thousands of different nucleic acids, such as detecting miRNA precursors and their targets, by Northern blot analysis or for addressing the spatiotemporal expression patterns of miRNA precursors and their targets including other non-coding RNAs or mRNAs by in situ hybridization in whole-mount embryos, whole-mount animals or plants or tissue sections of plants or animals, such as human, mouse, rat, zebrafish, Caenorhabditis elegans, Drosophila melanogaster, Arabidopsis thaliana, rice, and maize. Furthermore, the present oligonucleotide probes are useful for the capture and detection of naturally occurring or synthetic single stranded nucleic acids such as miRNA precursors and their targets not pre-sent in complex mixtures.

The present oligonucleotide probes of the invention are furthermore highly useful and applicable for large-scale and genome-wide expression profiling of miRNA precursors and their targets including other non-coding RNAs in animals and plants by oligonucleotide microarrays or small-scale expression profiling by RNA in-situ hybridization, dot blot hybridization, reverse dot blot hybridization, or in Northern blot analysis.

The present oligonucleotide probes are also useful for the purification of naturally occurring single stranded nucleic acids such as miRNA precursors and their targets.

Also featured is a method of creating a nucleotide duplex by providing a miRNA precursor; and contacting the miRNA precursor with a probe of the invention that hybridizes to the miRNA precursor, and the optional step of detecting the amount of a signal indicative of the miRNA precursor bound to the probe to quantify miRNA precursor in a sample.

The invention further features a method of comparing relative amounts of miRNA and miRNA precursor in a sample by contacting the sample with a first probe that hybridizes to miRNA precursor and a second probe that hybridizes to miRNA; and detecting the amount of one or more signals indicative of the relative amounts of miRNA and miRNA precursor.

The invention also features a method of measuring relative amounts of miRNA and miRNA precursor in a sample by contacting a first probe that hybridizes to miRNA with the sample under conditions that also allow miRNA precursor to hybridize; contacting the first probe or a second probe that hybridizes to miRNA with the sample under conditions that do not allow miRNA precursor to hybridize; comparing the amounts of the probes hybridized under the two conditions wherein the reduction in amount hybridized under the second condition compared to the first condition is indicative of the amount of miRNA precursor in the sample.

The invention also features methods of using the probes of the invention as components of Northern blots, in situ hybridization, arrays, and various forms of PCR analysis including PCR, RT-PCR, and qPCR.

Any probe of the invention may be used in performing any method of the invention. For example, any method of the invention may involve probes having labels. Furthermore, any method of the invention may also involve contacting a probe with miRNA precursor that is endogenously or exogenously produced. Such contacting may occur in vitro or in vivo, e.g., such as in the body of an animal, or within or without a cell, which may or may not naturally express the miRNA precursor.

Also, primarily with respect to miRNA precursors, nucleotide analogue containing probes, polynucleotides, and oligonucleotides are broadly applicable to antisense uses. To this end, the present invention provides a method for detection and functional analysis of non-coding antisense RNAs, as well as a method for detecting the overlapping regions between sense-antisense transcriptional units.

The invention also features a method of modulating, e.g., inhibiting or increasing, the biological activity of a miRNA precursor by providing the miRNA precursor; and contacting the miRNA precursor with a probe of the invention that hybridizes to the miRNA precursor, thereby inhibiting the biological activity of the miRNA precursor. In addition, the invention features a method of determining the biological activity of a miRNA precursor by providing the miRNA precursor; contacting the miRNA precursor with a probe of the invention that hybridizes to the miRNA precursor; and assaying the biological activity. The present oligonucleotide probes may be used in functional analysis of miRNA precursors and their targets including other non-coding RNAs in vitro and in vivo in plants or animals, such as human, mouse, rat, zebrafish, Caenorhabditis elegans, Drosophila melanogaster, Arabidopsis thaliana, rice and maize, by inhibiting their mode of action, e.g., the binding of miRNA precursor probes to their cognate target mRNAs. Suitable sources of target nucleic acid molecules include a wide range of eukaryotic and prokaryotic cells, including protoplasts; or other biological materials, which may harbor target nucleic acids.

The oligonucleotide probes of invention are also useful for detecting, testing, diagnosing or quantifying miRNA precursors and their targets implicated in or connected to human disease, e.g., analyzing human samples for cancer diagnosis.

As described in Example 5, pre-mir-138-2 is ubiquitously expressed, unlike its mature miRNA derivative. The presence of an unprocessed miRNA precursor in most tissues of the organism suggests miRNA precursors as possible diagnostic targets. We envision that miRNA precursor processing could be a more general feature of the regulation of miRNA expression and be used to identify underlying disease processes. One could also imagine that the unprocessed miRNA precursors might play a different role in the cell, irrespective of the function of the mature miRNA, providing further insights into underlying disease processes.

Imperfect processing of miRNA precursors to mature miRNA as detected by sample hybridization to oligonucleotide probes may provide diagnostic or prognostic information. Specifically, the ratio between levels of mature and precursor transcripts of a given miRNA may hold prognostic or diagnostic information. Furthermore, specific spatial expression patterns of mature miRNA compared to miRNA precursor may likewise hold prognostic or diagnostic information. In addition, performing in situ hybridization using mature miRNA and/or miRNA precursor specific oligonucleotide probes could also detect abnormal expression levels. LNA-containing probes are particularly well-suited for these purposes.

The present invention enables discrimination between different polynucleotide transcripts and detects each variant in a nucleic acid sample, such as a sample derived from a patient, e.g., addressing the spatiotemporal expression patterns by RNA in situ hybridization. The methods are thus applicable to tissue culture animal cells, animal cells (e.g., blood, serum, plasma, reticulocytes, lymphocytes, urine, bone marrow tissue, cerebrospinal fluid or any product prepared from blood or lymph) or any type of tissue biopsy (e.g., a muscle biopsy, a liver biopsy, a kidney biopsy, a bladder biopsy, a bone biopsy, a cartilage biopsy, a skin biopsy, a pancreas biopsy, a biopsy of the intestinal tract, a thymus biopsy, a mammae biopsy, a uterus biopsy, a testicular biopsy, an eye biopsy or a brain biopsy, e.g., homogenized in lysis buffer), archival tissue nucleic acids such as formalin fixated paraffine embedded sections of the tissue, plant cells or other cells sensitive to osmotic shock, and cells of bacteria, yeasts, viruses, mycoplasmas, protozoa, rickettsia, fungi and other small microbial cells and the like.

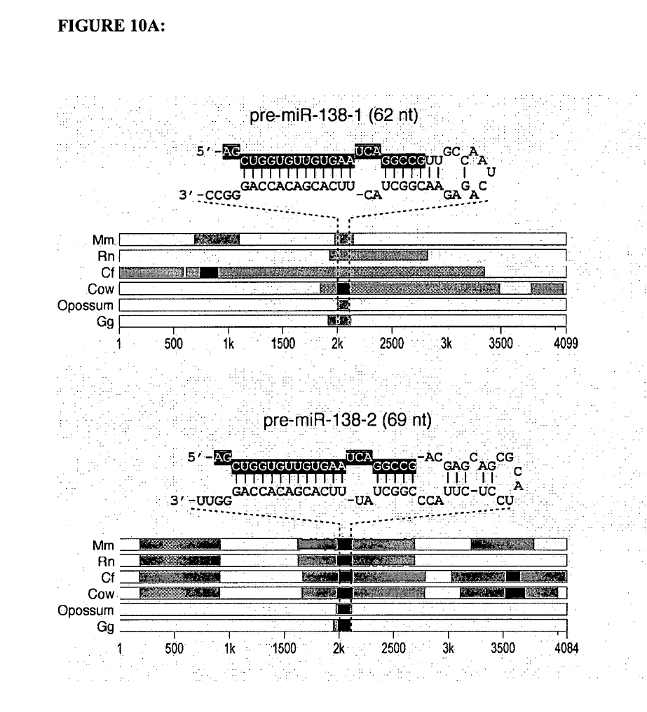

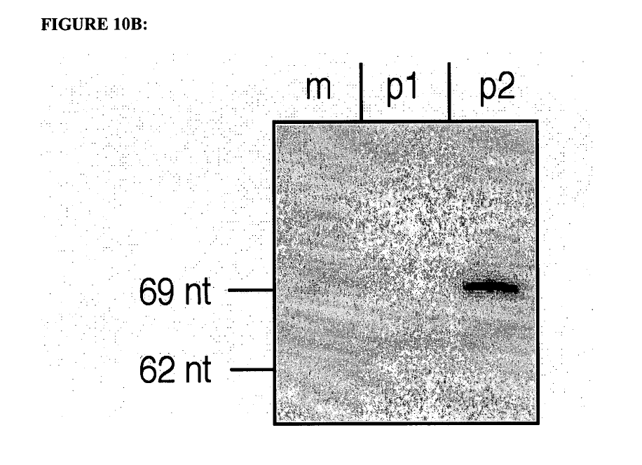

pre-mir-138-1 and pre-mir-138-2 and their shared mature miRNA derivative mir-138 differ in their expression levels across various tissues as detected by oligonucleotide probes. Examples 5 and 6 detail the differential expression of pre-mir-138-1 and pre-mir-138-2 and their derived mature miRNA mir-138. pre-mir-138-2 is expressed in all tissues (see also Examples 2 and 3), and mir-138 is expressed in a tissue-specific manner. Furthermore, the experiments suggest that an inhibitory factor is responsible for tissue-specific processing of pre-mir-138-2 into mir-138 and that this inhibitory factor is specific for certain miRNA precursors. This inhibitory factor acting on pre-138-2 may be capable of distinguishing pre-mir-138-1 from pre-mir-138-2 as well. pre-mir-138-1 and pre-mir-138-2 have different pre-mir sequences, particularly in the loop region, and thus the inhibitory factor may be capable of recognizing these sequence differences to achieve such specificity. It is hypothesized that recognition by an inhibitory factor is dependent on the differences in the loop sequence, e.g., the size of the loop sequence, between pre-mir-138-1 and pre-mir-138-2. It is therefore possible that an oligonucleotide probe capable of hybridizing specifically to the sequences that are different between pre-mir-138-1 and pre-mir-138-2, e.g. in the loop region, could be utilized to block the inhibitory effect of the inhibitory factor, thereby allowing the pre-mir-138-2 to be processed. Such a probe may have, for example, all or part of one of the sequences shown in FIG. 10A.

### EXAMPLES

### Example 1

### Detection of Mature miRNA and miRNA Precursors on Microarrays

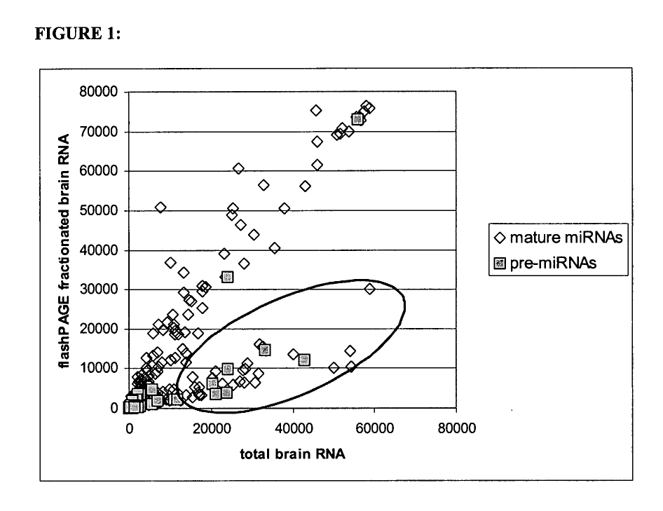

Experiment 1a: Mature miRNA probes bind both mature miRNA and longer transcripts, such as miRNA precursors, using oligonucleotide microarray. Total RNA pre-pared from human kidney, lung, and brain (Ambion) was hybridized to an array with and without sample filtering through a flashPAGE Fractionator (Ambion) which removes longer RNA transcripts, such as miRNA precursors, and retains shorter transcripts such as mature miRNA. In FIG. 1, the red oval identifies oligonucleotide probes whose signal is decreased after flashPAGE fractionation, including some oligonucleotide probes designed to target mature miRNA. This unexpected decrease in mature miRNA probe signal after fractionation suggests that longer transcripts, potentially miRNA precursors, are also binding the mature miRNA probes.

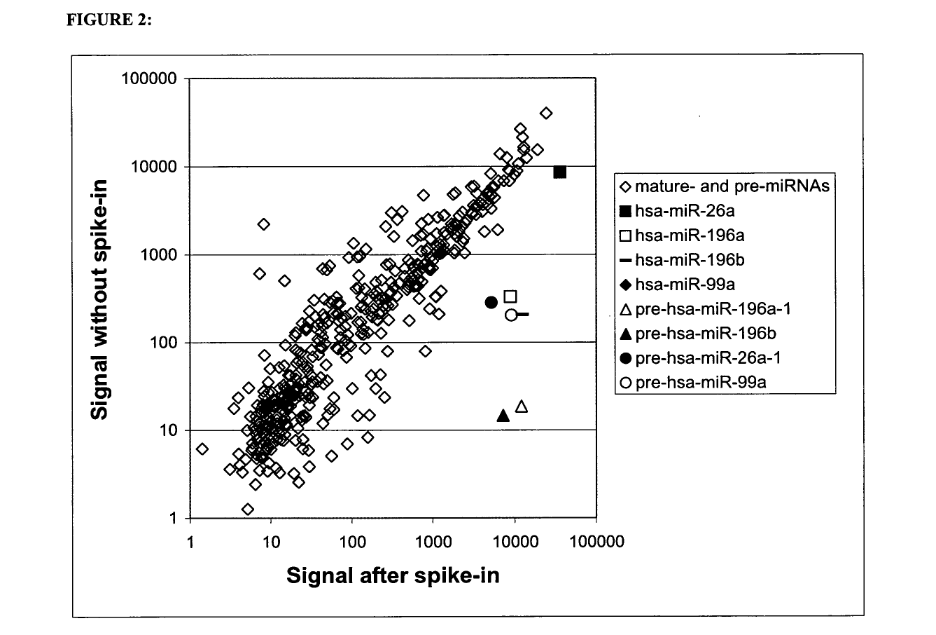

Experiment 1b: miRNA precursor is confirmed to bind mature miRNA probes. We next verified that the longer transcripts binding mature miRNA probes were miRNA precursors. This experiment was performed as Experiment 1a, except only total RNA from kidney was used, and to this RNA, 4 different in vitro-transcribed RNAs were added (so called spike-ins). These synthetic RNAs had a length of 62-65 nucleotides, and their sequence corresponded to 4 different pre-miRNA sequences (pre-hsa-miR-26a-1, pre-hsa-miR-99a, pre-hsa-miR-196a-1 and pre-hsa-miR-196b as listed in the Table 1). Results are shown in FIG. 2. The y-axis shows total kidney RNA without spike-in, and the x-axis shows the same experiment with 4 synthetic pre-miRNAs spiked-in to the total kidney RNA. Oligonucleotide probes for both the mature miRNAs and the pre-miRNAs have increased signal when the pre-miRNA is spiked in to the labeling reaction. (The oligonucleotide probe for mature hsa-miR-99a show no change due to a machine error, but visual inspection reveals that mature hsa-miR-99a does have an elevated signal after spike-in, consistent with the other probes.) The elevation in mature miRNA probe signal after spike-in confirms that miRNA precursors (pre-miRNA) are binding the mature miRNA probes.

TABLE 1

Sequences of spiked-in pre-miRNA in microarray experiment 1b

| | |
|---|---|
| Name | Sequence 5′ to 3′ |

| | |
|---|---|
| pre-hsa-miR-196a | GGGAGAtaggtagtttcatgttgttgggcctgggtttctgaacacaacaacattaaaccacccg |
| pre-hsa-miR-196b | GGGAGAtaggtagtttcctgttgttgggatccacctttctctcgacagcacgacactgcctt |
| pre-hsa-miR-26a | GGGAGAttcaagtaatccaggataggctgtgcaggtcccaatgggcctattcttggttacttgca |
| pre-hsa-miR-99a | GGGAGAaacccgtagatccgatcttgtggtgaagtggaccgcacaagctcgcttctatgggtc |

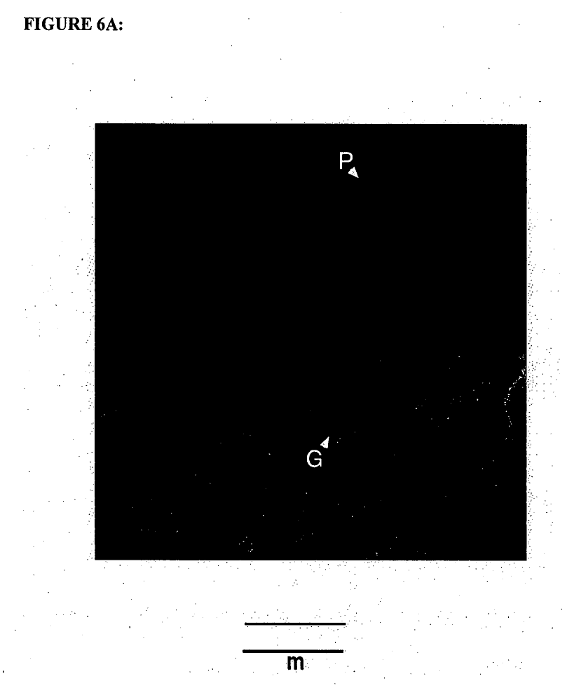

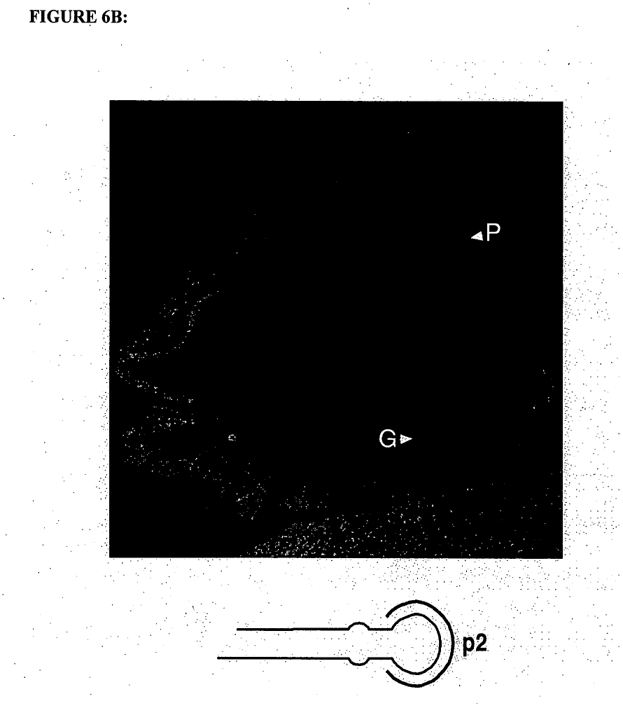

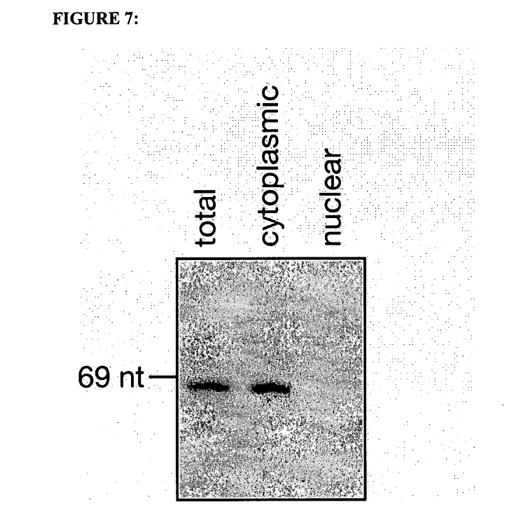

Furthermore, oligonucleotide probes designed to detect mature miRNAs also detected the pre-miRNAs only under high stringency conditions (as shown in FIG. 2). For array experiments, the low stringency condition is 60° C. in 30% FA (formamide), 4×SSC (Saline-Sodium citrate mixture) (approximate Tₘ of the hybridized probe is 72° C.). Under low stringency conditions, mature miRNA probes only bound mature miRNA because the miRNA precursors folded into hairpins, which are inaccessible under low stringency conditions. Furthermore, such low stringency conditions (55° C. hybridization and 60° C. wash) were used to perform the in situ hybridization experiments in Example 3 (FIGS. 6A, 6B, 7A, and 7B).

The selectivity of the mature miRNA probes under different conditions can be exploited to differentiate signals from mature miRNA and pre-miRNA by performing sample hybridization to mature miRNA probes under two different conditions: under high hybridization stringency, both mature miRNA and miRNA precursor will hybridize to the mature miRNA probes, and under low stringency, the pre-miRNA will not hybridize to the mature miRNA oligonucleotide probe; thus, the difference in hybridization between the two conditions corresponds to the amount of miRNA precursor.

Stringency in hybridization experiments is typically controlled by temperature and buffer composition (salt—such as NaCl—concentration and formamide concentration). One mathematical equation describing the relationship between the different components in determining the Tₘ of a hybridized oligonucleotide probe is given below (Nucleic Acid hybridization, Ed B. D. Hames & S. J. Higgins IRL Press, 1985):

Tₘ=81.5+16.6(log M)+0.41*(%G+C)−0.72(% formamide)

where M is the molar concentration of monovalent cations,

% G+C is the percentage of guanine and cytosine residues in the DNA,

% formamide is the volume percent of formamide

Therefore high and low stringency conditions can be adjusted by manipulating hybridization temperature, salt, and formamide concentration in the hybridization buffer.

High stringency conditions for pre-miR detection can be defined as 72° C. or 69° C., or 66° C., while low stringency conditions include 55° C., 58° C., 61° C. or 63° C.

Taken together, these experiments demonstrate that mature miRNA probes on a microarray can be used to detect miRNA precursors (pre-miRNAs) in a sample.

Furthermore, these experiments demonstrate that oligonucleotide probes designed to detect the pre-miRNA form (loop part) of the transcripts can detect pre-miRNA transcripts in human total RNA. This is demonstrated in FIG. 2 where the oligonucleotide probes detecting pre-miRNA loop regions get increased signal after synthetic pre-miRNA spike-in.

Additionally, the ratio between the mature miRNA and miRNA precursor probes, hybridized under only one stringent condition, can be used to estimate the amount of pre-miRNA and mature miRNA in a sample.

### Methods:

Microarray production. 84 miRNAs were selected from previous experiments based on 2 criteria. Either they had an interesting pattern on a microarray with total-RNA and flashPAGE enriched RNA (FIG. 1), or there were other indications that these miRNAs might exist in their unprocessed pre-miRNA form. Oligonucleotide probes were designed for the loop region of these pre-miRNAs and these were spotted on the microarray in parallel with the mature miRNA oligonucleotide probes (miRCURY ready-to-spot oligonucleotide probes version 7.1). All probes were designed to have a Tₘ of 72° C. and to include both DNA and LNA monomers.

Microarrays were spotted on CodeLink slides (Amersham Biosciences) in miRCURY standard spotting buffer (300 mM NaPO4, pH 9.0, 0.001% Triton-X) at a concentration of 20 μM. Post-processing of slides was performed according to the slide manufacturer's instructions (CodeLink user guide, Amersham Biosciences).

Fractionation and Microarray Hybridization. Total RNA prepared from human kidney, lung and brain (Ambion) was either labeled directly or after running samples through a flashPAGE Fractionator (Ambion), known to enrich small RNAs and remove longer transcripts. For direct labeling, 2 μg of total RNA was used, and total RNA samples were labeled with Hy3. 30 μg of total RNA was subjected to flashPAGE fractionation and flashPAGE reaction cleanup according to the manufacturer's protocols and finally eluted in 30 μl. ⅓ of the eluted volume was labeled with Hy5 and combined with Hy3-labeled total RNA from the same tissue. All labeling reactions were performed using the miRCURY array labeling kit according to standard Exiqon protocols.

After combining Hy3 (total RNA) and Hy5 (flashPAGE fractionated RNA) from the same tissue, samples were concentrated in a speedvac at room temperature. Labeled sample volume was adjusted to 25 μl, and mixed with 25 μl miRCURY hybridization buffer. The sample was hybridized onto the spotted and preprocessed slide in a hybridization machine (Tecan) and hybridized at 60° C. for 16 hours according to standard Exiqon protocols. Washing and drying were also performed in the hybridization machine according to Exiqon user manual instructions.

Four different in vitro-transcribed RNAs were added to total RNA from kidney cells (so called spike-ins). These synthetic RNAs had a length of 62-65 nucleotides and their sequence corresponded to 4 different pre-miRNA sequences (pre-hsa-miR-26a-1, pre-hsa-miR-99a, pre-hsa-miR-196a-1 and pre-hsa-miR-196b as listed in the Table 1). The synthetic RNAs were added at a concentration of 2.5 fmol each to 1 μg total RNA sample (130 fmol each to 52 μg total RNA). Again, 2 μg of this RNA was labeled directly and 30 μg was fractionated using flashPAGE. Total and flashPAGE-fractionated RNAs were labeled and hybridized onto slides from the same printing batch.

Microarray Image and Data Analysis. Slides were scanned using a ScanArray scanner (Perkin Elmer) and images were analyzed using Imagene software (BioDiscovery). The signal from each oligonucleotide probe was calculated as the signal minus local background, and whenever two channels were compared, the total intensity was normalized to be equal. As each oligonucleotide probe was spotted 4 times on each microarray, an average of these measurements was used for each probe.

### Example 2

### Tissue Specificity of miRNA Precursors as Measured by Northern Blot

As a first step in validating targets of a set of brain-specific miRNAs and miRNA precursors, we isolated total RNA from brain and other mouse tissues, as well as from murine N2A neuroblastoma and HeLa cells, and performed Northern blots with miRNA-specific and miRNA precursor-specific probes.

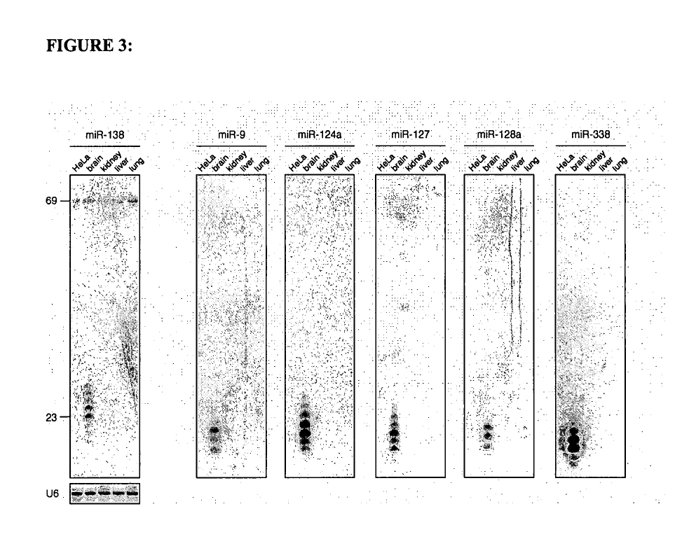

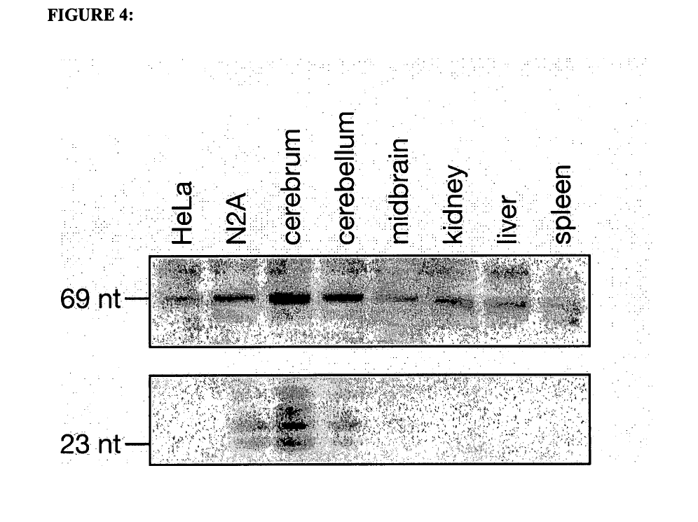

Surprisingly, in the case of miR-138, we observed a band of ˜70-nt corresponding to its precursor, pre-miR-138, which was present in all tissues and cells analyzed (FIG. 3). In contrast, the mature ˜23-nt miR-138 was detectable only in the cerebrum and cerebellum of adult mice as well as in N2A cells (FIG. 4), suggesting that the ubiquitously expressed precursor is processed into the mature miRNA in a tissue specific manner. Other miRNA like miR-9, miR124a, miR-127, miR-128a and miR228 are pre-sent only as mature forms in all tissues analyzed (FIG. 3).

### Methods:

Isolation of total RNA from cells and mouse tissues and Northern blotting. Isolation of total RNA from cultured cells or tissues and subsequent Northern blotting was performed as previously described (Lagos-Quintana et al., Science 294:853-858, 2001). 20 μg of total RNA was separated in a 15% polyacrylamide gel (20×25 cm) containing 8 M urea (SEQUAGEL, National Diagnostics), transferred to a Hybond-N+ membrane (Amersham Biosciences), fixed by ultraviolet cross-linking (2% auto crosslink on a Stratalink 2400, Stratagene) and subsequently baked for 1 h at 80° C. Membranes were probed with 10 pmoles of 5′ 32P-labeled (T4 polynucleotide kinase, New England Biolabs) DNA/LNA (locked nucleic acid) oligonucleotides (Proligo, France), complementary to the mature and precursor miRNAs. We used DNA/LNA probes, where every third position was substituted by a LNA monomer, in order to obtain an improved miRNA detection (Valoczi et al., Nucleic Acids Res. 32:e175, 2004). LNA monomers are indicated by ‘*X’. The sequences for the probes were as follows: mmu-miR-138: 5′-C*GGC*CTG*ATT*CAC*AAC*ACC*AGC*T. To differentiate between miRNA precursors and mature miRNAs, the following probe against the loop region of the hairpin was designed and labeled: pre-miR-138-2: 5′-G*GTA*AGA*GGA*TGC*GCT*GCT*CGT. Pre-hybridization of membranes was carried out in a buffer containing 5×SSC (Saline-Sodium citrate mixture: 175.3 gram of NaCl and 88.2 gram of sodium citrate in 1 L of distilled water), 20 mM NaH₂PO₄ (pH 7.2), 7% SDS (Sodium Dodecyl Sulfate), 1% Denhardt's solution and 0.1 mg/ml sonicated salmon sperm DNA (Stratagene). Hybridizations were carried out in the same solution at 80° C. 5′-32P-labeled probes were heated for 1 min at 95° C. before addition to the hybridization solution. After hybridization, the membranes were washed twice in 5×SSC, 5% SDS and once in 1×SSC, 1% SDS at 70° C. for 1-2 min each. Northern blots were then analyzed by phosphorimaging (Storm 860, Molecular Dynamics).

Cell Culture. HeLa human cervix-carcinoma cells as well as N2A murine neuroblastoma cells were grown in Dulbecco's modified Eagle's medium (DMEM, Invitrogen) supplemented with 10% fetal calf serum (FCS, Invitrogen), 100 u/ml penicillin (Sigma-Aldrich), 100 μg/ml penicillin/streptomycin (Sigma-Aldrich) and 20 mM HEPES pH 7.3 (4-(2-hydroxyethyl)-1-piperazineethanesulfonic acid) at 37° C. in an atmosphere containing 5% CO₂ in 15 cm-dishes up to 90-95% confluency.

Preparation of Cellular Extracts. HeLa or N2A cells were briefly washed with 1×PBS (Phosphate-Buffered Saline, Invitrogen Inc.) and harvested them in 1 ml of a buffer containing 100 mM KCl, 5 mM MgCl₂, 10% glycerol, 30 mM HEPES (pH 7.4), 0.1 mM AEBSF (4-(2-Aminoethyl)benzenesulphonyl fluoride), 0.5 mM DTT (dithiothreitol) by scraping with a rubber policeman. Cells were effectively lysed by sonication. The remaining cell debris was removed by centrifugation. Nuclear and cytoplasmic extracts were prepared as previously described (Lehnertz et al., Curr. Biol. 13:1192-1200, 2003). Briefly, to prepare cytoplasmic extracts, cells were harvested by trypsinization, washed and pelleted by centrifugation. The pellet was resuspended in 1×PBS and pelleted again by spinning. PBS was removed and the cell pellet was resuspended in cold buffer A, containing 10 mM HEPES [pH 7.9], 10 mM KCl, 0.1 mM EDTA (ethylenediaminetetraacetic acid), 0.1 mM EGTA (ethylene glycol bis(2-aminoethyl ether)-N,N,N′N′-tetraacetic acid), 1 mM DTT, 0.5 mM PMSF (Phenylmethylsulphonylfluoride), by gentle pipetting. After swelling, a 10% solution of Nonidet NP-40 (Fluka) was added, and the tube was vigorously vortexed. The homogenate was centrifuged, and the supernatant containing the cytoplasm and the cytoplasmic RNA was transferred to a fresh tube and subsequently used for RNA extraction and Northern blot analysis. Nuclei were pre-pared by centrifugation of the cells through a cushion of nuclear isolation buffer (20% [v/v] Ficoll-Paque Pharmacia, 80 mM Tris/HCl [pH 7.4], 8 mM MgCl₂, 8 mM CaCl₂, 1.6% [v/v] Nonidet NP-40, 1.3% [v/v] Triton X-100, and 0.001% [v/v] DMSO). Nuclear pellets were washed once in ice-cold PBS, snap frozen in liquid nitrogen, and subsequently used for the preparation of nuclear RNA. To obtain nuclear extracts, nuclei were resuspended in immunoprecipitation (IP) buffer (45 mM HEPES/NaOH [pH 7.5], 0.45 M NaCl, 0.9 mM EDTA, 0.9% [v/v] NP-40, 8.7% [v/v] glycerol, 1 mM NaF, 10 mM β-glycerophosphate, 1 tablet/50 ml protease inhibitor cocktail Roche #1836145) and sonicated. After centrifugation, the supernatant was snap frozen in liquid nitrogen and subsequently used for RNA extraction and Northern blot analysis.

### Example 3

### Detection of Pre-miRNAs In Situ Using DIG-Labeled Oligonucleotides

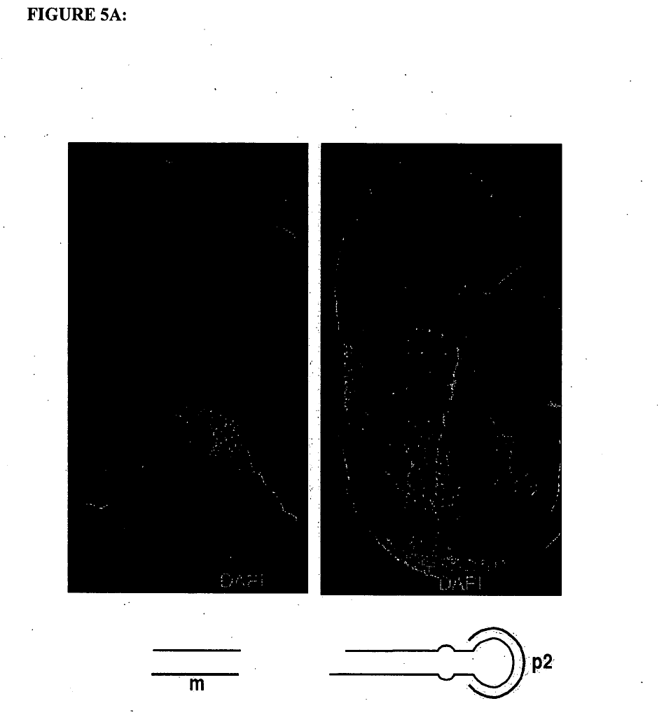

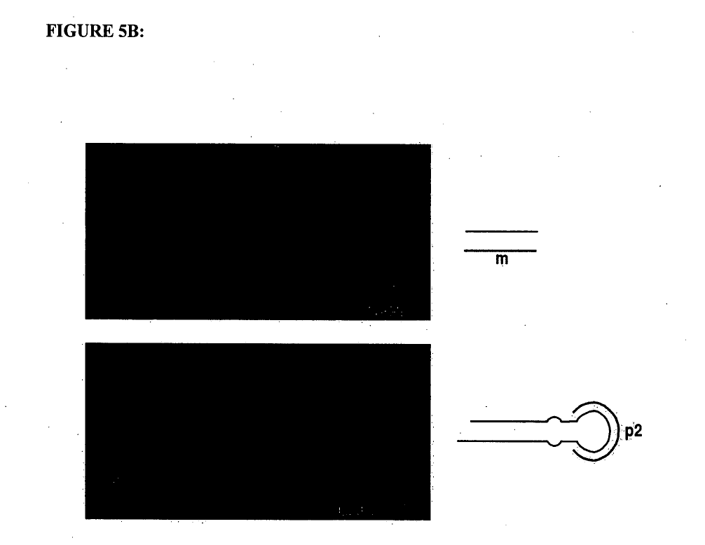

To further investigate the overall distribution of miR-138 and its precursor, we performed in situ hybridizations with 3′ DIG-labeled LNA oligonucleotide probes on cryo-sections of E17 mouse embryos (FIG. 5A) and adult brain (FIG. 5B). The sequences of the oligonucleotide probes were as follows (capital letters indicate LNA and mC indicates methyl cytosine):

| | |
|---|---|
| miR-122a: | 5′-acAaamCacmCatTgtmCacActmCca-3′ |
| miR-138: | 5′-gatTcamCaamCacmCagmCt-3′ |
| pre-miR-138-2: | 5′-ggtAagAggAtgmCgcTgcTcgt-3′ |

We observed a strong staining in the central nervous system (CNS) for miR-138 (FIG. 5A, left panel). In particular, miR-138 was primarily localized to Purkinje and granule cells of the cerebellum, but also to most neurons in the hippocampus and to specific regions of the neocortex (FIG. 5A, left panel, and FIG. 6A). This clearly demonstrates that expression of miR-138 is not uniform throughout the brain but restricted to distinct cellular layers. Surprisingly, miR-138 was expressed in fetal (FIG. 5A, left panel) but not in adult liver (FIG. 5B), indicating that the expression of miR-138 is regulated both in time and space. To confirm the ubiquitous expression observed for premiR-138-2 in Northern blots with total RNA from adult tissues, we prepared a probe recognizing the loop region of the pre-miR-138-2 hairpin. In situ hybridizations showed a broad expression of pre-miR-138-2 in most organs of the embryo with the exception of the brain (FIG. 5A, right panel, FIG. 6B), which might be explained by efficient processing of the precursor by Dicer, which ultimately leads to its depletion in these regions.

Under the hybridization conditions used, the probe detecting the mature miR-138 (probe m in FIG. 5B) does not detect the pre-miR138-2, probably because the target (the mature miR-138 sequence) is inaccessible to hybridization due to the hairpin structure of the pre-miR. This feature is consistent with previous observations about the specificity of mature miRNA probes for mature miRNA under certain stringent conditions.

### Methods:

In situ hybridizations. C57BL/6 mice were mated to generate embryos for analyses and the morning of the vaginal plug was considered as E0.5. Embryos, livers, and brains were postfixed in 4% paraformaldehyde (PFA), cryoprotected (30% sucrose in PBS), embedded in Tissue-Tek OCT compound and cryosectioned. 10 μm cryosections were pre-treated, hybridized with LNA digoxigenin-labeled probes, and washed according to Schaeren-Wiemers and Gerfin-Moser (Schaeren-Wiemers & Gerfin-Moser, Histochemistry 100:431-440, 1993), with some modifications. The sequences of the oligonucleotide probes were as follows:

| | |
|---|---|
| miR-122a: | 5′-acAaamCacmCatTgtmCacActmCca-3′ |
| miR-138: | 5′-gatTcamCaamCacmCagmCt-3′ |
| Pre-miR-138-2: | 5′-ggtAagAggAtgmCgcTgcTcgt-3′ |

Briefly, sections were fixed in 4% paraformaldehyde for 10 minutes, acetylated and treated with 5 μg/ml proteinase K (Roche) in PBS for 5 minutes, washed, and prehybridized for 4 h at room temperature. Hybridization with 22-nt LNA probes (Tₘ˜80° C.) was performed at 55° C. overnight. Slides were then washed at 60° C. and incubated with alkaline phosphatase (AP)-conjugated goat anti-digoxigenin Fab fragments (Roche, 1:2000) at 4° C. overnight. Fluorescent detection was performed using 1 hr incubations with Alexa Fluor 488 goat anti-rabbit IgG (Molecular Probes, 1:500) followed by a Fast Red reaction (Dako Cytomation) for 1 h at room temperature. Sections were analyzed with a with a Zeiss Axioplan-2 microscope and photographed with a digital camera (Photometrics, Coolsnap HQ). A subset of images was adjusted for levels, brightness, contrast, hue, and saturation with Adobe Acrobat 7.0 imaging software to optimally visualize the expression patterns.

### Example 4

### Oligonucleotide Probes for miRNA Precursor Detection on Microarrays

Table 2 shows sequences of the oligonucleotide probes designed for measuring microRNA precursors. These oligonucleotide probes were spotted onto microarrays used in Example 1 to detect microRNA precursors. Capital letters indicate LNA and mC indicates methyl cytosine.

The oligonucleotide probes have sequences complementary to the loop-region of the pre-miRNA indicated in the probe name. A stretch of 25 nucleotides was identified in the center of the loop-region of each miRNA precursor, and an oligonucleotide probe was designed complementary to each 25-mer sequence. This design process takes into account the predicted Tₘ of the hybridized oligonucleotide probe, self-annealing of the probe to identical probes and possible intra-molecular secondary structures, and the desired difference between Tₘ and self-annealing Tₘ. A similar design process is used to create probes that hybridize to mature miRNA. Furthermore, the design of the probe is constrained to enhance the yield of their synthesis, e.g., probes without LNA residues in the 3′-end can be synthesized with higher yields. Oligonucleotide probe sequences are selected to have the greatest possible specificity for their intended miRNA precursors (to the exclusion of other miRNA precursors and miRNAs) to enhance the probes' ability to discriminate between different miRNA precursors.

TABLE 2

| | |
|---|---|
| Name | Sequence |

| | |
|---|---|
| 22691_pre-hsa-miR-1-1 | ttmCttTacAttmCcaTagmCttAgcAg |
| 22692_pre-hsa-miR-1-2 | amCttmCttTacAttmCcaTagmCatTgt |
| 22693_pre-hsa-miR- | aTggmCgtTtgAtaGttTagAcac |
| 122a | |
| 22694_pre-hsa-miR- | cAccGcgTgcmCttAat |
| 124a-1 | |
| 22695_pre-hsa-miR- | tTgtAtgAcaTtaAatmCaaGgtmCc |
| 124a-2 | |
| 22696_pre-hsa-miR- | gcGtgmCctTaaTtgTatAgamCatt |
| 124a-3 | |
| 22697_pre-hsa-miR-127 | tcmCgaTgaTctTtcTgaAtcAg |
| 22698_pre-hsa-miR- | mCacTgtGagAaaTgtAaamCctc |
| 128a | |
| 22699_pre-hsa-miR- | AagGgcTtcmCtgActActGt |
| 129-1 | |
| 22700_pre-hsa-miR- | GgcTtcmCggmCtaTtgAg |
| 129-2 | |
| 22701_pre-hsa-miR- | caTtgmCacTgcTtcmCca |
| 130b | |
| 22702_pre-hsa-miR-134 | mCacAggGtgAacAcaGt |
| 22703_pre-hsa-miR- | tcTgaTtgGcaAcgGcc |
| 138-1 | |
| 22704_pre-hsa-miR- | cGggTaaGagGatGc |
| 138-2 | |
| 22705_pre-hsa-miR-147 | agAagmCatTtcmCacAcamCtg |
| 22706_pre-hsa-miR-15 | ggmCctGcamCctTttmCaa |
| a | |
| 22707_pre-hsa-miR-16- | aGatAatTttAgaAtcTtaAcgmCcaAt |
| 1 | |
| 22708_pre-hsa-miR-16- | AgtAatAttGgtGttTaaTatAt |
| 2 | |
| 22709_pre-hsa-miR-184 | gTccAacActTacAgtmCaca |
| 22710_pre-hsa-miR-185 | tgGggAggGgac |
| 22711_pre-hsa-miR-187 | mCagAgcAgcGccc |
| 22712_pre-hsa-miR- | tgTtgTgtTcaGaaAccmCag |
| 196a-1 | |
| 22713_pre-hsa-miR- | AgtTtcTtgTtgmCcgAgtTcaAa |
| 196a-2 | |
| 22714_pre-hsa-miR- | cgAgaGaaAggTggAtcc |
| 196b | |
| 22715_pre-hsa-miR-198 | taTagAgaAgaAggAaaAatmCacAgga |
| 22716_pre-hsa-miR- | cAcaTtgAgaGccTccTg |
| 199a-1 | |
| 22717_pre-hsa-miR- | aAcgGcaTtgTccTgaAcag |
| 199a-2 | |
| 22718_pre-hsa-miR-19 | ttGcamCaamCtamCatTctTctTgt |
| a | |
| 22719_pre-hsa-miR- | acAgcTggAtgmCaaAcc |
| 19b-1 | |
| 22720_pre-hsa-miR- | tAcaTatAtamCgcTgaAatGcaAac |
| 19b-2 | |
| 22721_pre-hsa-miR- | gcAgtAttAgaGacTccmCa |
| 200c | |
| 22722_pre-hsa-miR-202 | cTttAggmCcaGatmCctmCaaa |
| 22723_pre-hsa-miR-203 | TcamCaaTtgmCgcTacAgaAc |
| 22724_pre-hsa-miR-21 | GccAtgAgaTtcAacAgtmCa |
| 22725_pre-hsa-miR-214 | ggTgaGcgGatGttmCtg |
| 22726_pre-hsa-miR-24- | ttmCctGctGaamCtgAgc |
| 1 | |
| 22727_pre-hsa-miR-24- | tgTacAcaAacmCaamCtgTgtTtc |
| 2 | |
| 22728_pre-hsa-miR- | cAttGggAccTgcAc |
| 26a-1 | |
| 22729_pre-hsa-miR- | mCctmCacAgaTggAaamCag |
| 26a-2 | |
| 22730_pre-hsa-miR-324 | cAgcTttAcamCcaAtgmCc |
| 22731_pre-hsa-miR-325 | cTcaAtaAacAaaTtaTgtmCacAaamCa |
| 22732_pre-hsa-miR-326 | AtcTgaGcamCcamCccg |
| 22733_pre-hsa-miR-328 | ggmCtgTatGcamCttTctmCc |
| 22734_pre-hsa-miR-331 | mCtgGttTgaTctGggAtcc |
| 22735_pre-hsa-miR-338 | gTcgmCctGagTcaTca |
| 22736_pre-hsa-miR-346 | tGccTccTtcAgaGcaAc |
| 22737_pre-hsa-miR-370 | mCgtGagmCtgTgtAacTg |
| 22738_pre-hsa-miR-373 | mCccAgtAcaGacAaaAagGaa |
| 22739_pre-hsa-miR-375 | AcaAaamCgcTcaGgtmCc |
| 22740_pre-hsa-miR-381 | tmCcaTgtmCaaTaaAccGaaTatAca |
| 22741_pre-hsa-miR-382 | tGatTcgTcaTaaGtaAagmCga |
| 22742_pre-hsa-miR-409 | AgcAacAttmCgtmCgtmCc |
| 22743_pre-hsa-miR-423 | tTttGgaAaaTagAaaAgtmCtcGct |
| 22744_pre-hsa-miR-432 | cmCacGtaAggAaaTagAggAtc |
| 22745_pre-hsa-miR-433 | amCacAgaGgaTctAgc |
| 22746_pre-hsa-miR-452 | amCtgAgamCatAgtTacAaaGtc |
| 22747_pre-hsa-miR-485 | tGacTcgmCttTgaTgaAtcg |
| 22748_pre-hsa-miR-492 | tGgcGttmCtcAatGg |
| 22749_pre-hsa-miR-494 | tgTttmCatmCatAaaTaaAgaGaaGaca |
| 22750_pre-hsa-miR-498 | gAggTgcTttTcaTccAgt |
| 22751_pre-hsa-miR-500 | aTtcAgamCagmCacTctmCa |
| 22752_pre-hsa-miR-503 | mCccAgaGcamCcgAtc |
| 22753_pre-hsa-miR-510 | gTttmCaaTcamCacmCtaAttAcaTg |
| 22754_pre-hsa-miR- | amCttTcaTtcTggmCacmCa |
| 512-1 | |
| 2755_pre-hsa-miR- | acAgcActTtcAttmCtgGca |
| 512-2 | |
| 22756_pre-hsa-miR- | GgtGaaAttTatAttTtaGttmCacAt |
| 513-1 | |
| 22757_pre-hsa-miR- | acTctGctTtcAgamCaamCag |
| 515-1 | |
| 22758_pre-hsa-miR- | AgcActTtcTttTctTtcAgamCaamCa |
| 516-1 | |
| 22759_pre-hsa-miR- | gAagmCacTttmCttTcamCaaAacAcaa |
| 516-3 | |
| 22760_pre-hsa-miR- | ggAagmCacTttmCttTtcTttmCac |
| 516-4 | |
| 22761_pre-hsa-miR- | gmCttTctmCttmCttTtaGacAacAga |
| 518a-2 | |
| 22762_pre-hsa-miR- | AgcGctTtgTttTctTtcAgamCa |
| 518c | |
| 22763_pre-hsa-miR- | AgcGctTtcTttTctTttAgamCaag |
| 518f | |
| 22764_pre-hsa-miR- | tgTttAagAgaAaamCaaAcaGaaAg |
| 519d | |
| 22765_pre-hsa-miR- | cActTtgTttTctTtcAaamCagAaag |
| 519e | |
| 22766_pre-hsa-miR- | cgmCctTctTttTttTcamCatAagAg |
| 525 | |
| 22767_pre-hsa-miR- | cgmCttTctmCttmCttTcaAgcAaca |
| 526a-1 | |
| 22768_pre-hsa-miR- | tgmCatGttmCttTtcTttmCaamCagAa |
| 526a-2 | |
| 22769_pre-hsa-miR- | aAgcActTtcTctTctTtcAgamCaac |
| 526b | |
| 22770_pre-hsa-miR-527 | ActTtcTttTctTttAgamCaamCaga |
| 22771_pre-hsa-miR-9-1 | agActmCcamCacmCacTcat |
| 22772_pre-hsa-miR-9-2 | atGaaGacmCaaTacActmCatAca |
| 22773_pre-hsa-miR-9-3 | tTatGacGgcTctGtgg |
| 22774_pre-hsa-miR-99 | agGtcmCacTtcAccAcaAga |

### Example 5

### An Inhibitory Factor is Responsible for Sequence and Tissue Specific Processing of Pre-miR-138

We further explored the cause of differential miRNA precursor processing into mature miRNA across tissues. The export of pre-miR-138-2 may be impaired in all tissues except brain, thus preventing cleavage by Dicer in the cytoplasm. To evaluate this possibility, we isolated RNA from the cytoplasm (where pre-miRNA is processed) and nucleus of HeLa cells (where pre-miRNA is not processed), and examined its sub-cellular distribution. Northern blot analysis showed that the precursor is effectively exported to the cytoplasm (FIG. 7), indicating that cleavage by Dicer is the regulated step. The sequences used were as follows:

pre-miR-19a,
TCAGTTTTGCATAGATTTGCACAACTACATTCTTCTTGTAGTGCAACTAT
GCAAAACCTATAGTGAGTCGTATTAA;
pre-miR-138-2,
AACCCTGGTGTCGTGAAATAGCCGGGTAAGAGGATGCGCTGCTCGTCGGC
CTGATTCACAACACCAGCCTATAGTGAGTCGTATTAA.

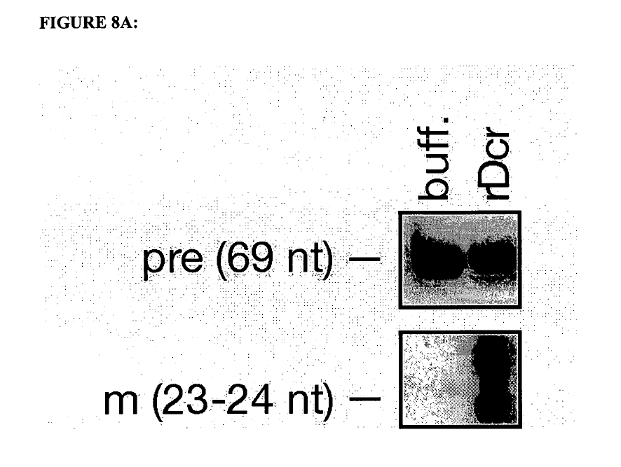

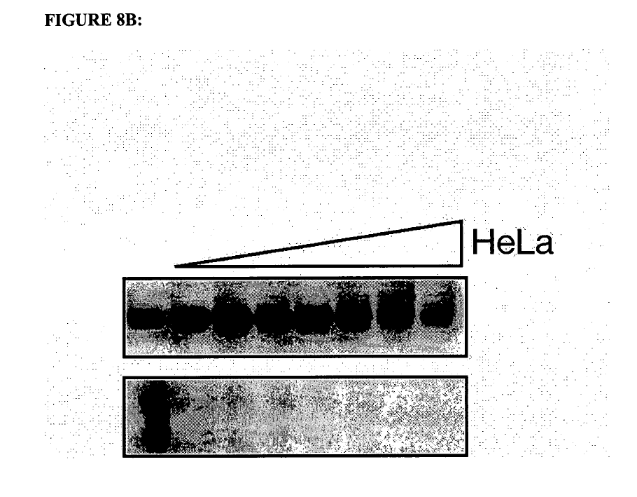

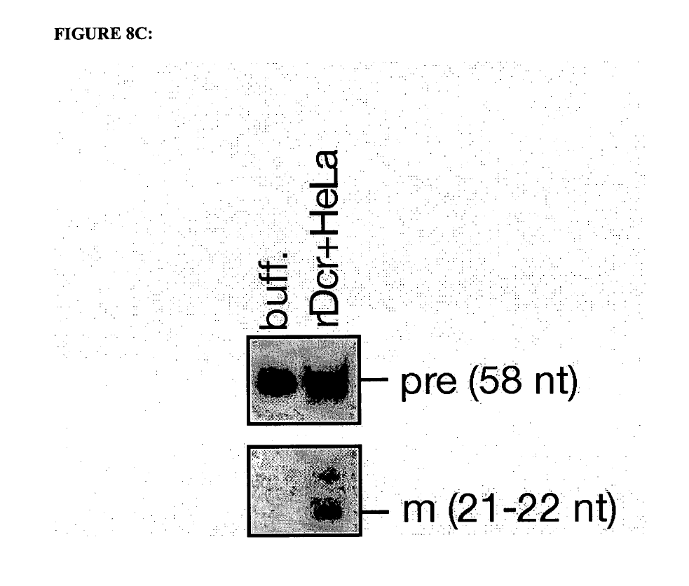

In this scenario, tissue-specific expression could be achieved either by an activator present on those tissues that express miR-138, or alternatively by an inhibitor acting on tissues that lack expression of miR-138. We were able to show that in an in vitro processing assay, recombinant Dicer enzyme is able to convert pre-miR-138-2 into mature miR-138, a result that rules out the necessity of an activator (FIG. 8A). This leaves the remaining possibility that the presence of an inhibitory factor, acting in all tissues not expressing mature miR-138, binds pre-miR-138-2 and prevents its conversion into a mature miR-138 by Dicer. This hypothesis is fostered by in vitro experiments, where the addition of increasing amounts of HeLa cytoplasmic extracts specifically impaired processing of pre-miR-138-2 by recombinant Dicer (FIG. 8B). Importantly, processing of premiR-19a, a miRNA that is normally expressed in HeLa cells (Lagos-Quintana et al., Science 294:853-858, 2001), was not impaired in a similar assay (FIG. 8C). This suggests that HeLa cells, and possibly all other tissues and cells that do not express mature miR-138, may contain a factor that specifically recognizes pre-miR-138-2 and inhibits its processing by Dicer.

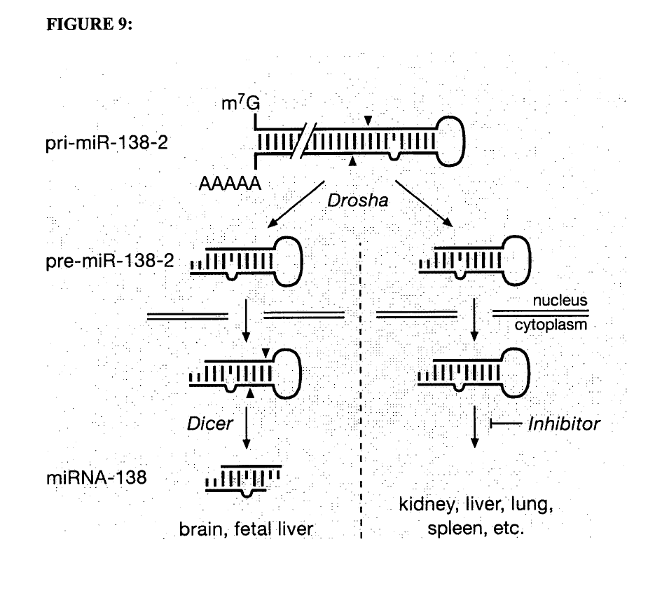

Thus, tissues and cells that express miR-138 may lack this inhibitory factor or may render it inactive allowing Dicer cleavage to occur (FIG. 9). Therefore, the differential processing of precursor miRNAs into mature miRNAs leads to tissue- and developmental specific miRNA expression in mammals, and therefore detection of pre-miRNAs as well as mature miRNA is important for understanding the function of microRNA in development and disease. Supporting this conclusion, it has been shown previously in c. elegans that miR-38 is regulated in a temporal manner by differential maturation of pre-miR-38 during development (Ambros et al., Curr. Biol. 13:807-818, 2003). The presence of an unprocessed miRNA precursor in most tissues of the organism is intriguing. One could imagine that the unprocessed precursor might play a different role in the cell, irrespective of the function of the mature miRNA. We envision that this novel mechanism could be a more general feature for the regulation of miRNA expression.

### Methods:

In vitro processing assays using Northern blotting. For in vitro processing assays, DNA templates encoding the sequences of various miRNAs were transcribed by T7 polymerase (MEGAshortscript™ T7, Ambion) in the presence of 32P-α-UTP (Amersham) thus generating radiolabeled precursor miRNAs. The sequences used were as follows:

pre-miR-19a,
TCAGTTTTGCATAGATTTGCACAACTACATTCTTCTTGTAGTGCAACTAT
GCAAAACCTATAGTGAGTCGTATTAA;
pre-miR-138-2,
AACCCTGGTGTCGTGAAATAGCCGGGTAAGAGGATGCGCTGCTCGTCGGC
CTGATTCACAACACCAGCCTATAGTGAGTCGTATTAA.

These synthetic precursors were folded into their hairpin-shaped structure by heating for 1 min at 95° C. and cooling slowly to room temperature. Processing with recombinant Dicer was performed as previously described (Zhang et al., EMBO J 21:5875-5885, 2002). Precursors were used at a concentration of 10 nM and were pre-treated for 10 min at 30° C. with increasing amounts of HeLa cytoplasmic extract (2 μg, 4 μg, 6 μg, 8 μg, 16 μg, 24 μg and 32 μg protein for pre-miR-138-2, and 32 μg protein for pre-miR-19a). The reaction products were separated on a 15% denaturing PAGE and visualized by autoradiography.

### Example 6

### Identification of Chromosomal Origin of Pre-miRNAs with Chromosome-Specific Probes

MicroRNAs precursors can be expressed from multiple locations even on different chromosomes. For example, two putative genes were predicted to encode miR-138 in chromosomes 8 and 9 (Lagos-Quintana et al., Curr. Biol. 12:735-739, 2002; Weber, Febs. J. 272:59-73, 2005). The predicted precursors of miR-138 are termed miR-138-1 and miR-138-2 (previously named miR-138) (Griffiths-Jones, Science 93:834-838, 2004), which are 62 and 69-nt in size, respectively. Multiple sequence alignments among different vertebrate species (Schwartz et al., Genome Res. 10:577-586, 2000) showed high conservation of the mature miRNA sequence for both precursors, whereas pre-miR-138-2, but not pre-miR-138-1 showed an overall conservation pattern even in the flanking regions up- and downstream of the encoded miRNA (FIG. 10A). Interestingly, we never detected expression of pre-miR-138-1 by Northern blotting (FIG. 10B). These results, together with the observed size (69-nt) in Northern blots, suggest that the miR-138 in HeLa cells is derived from pre-miR-138-2. Therefore probes targeting the loop region of pre-miR-138-2 and pre-miR-138-1 can be used to identify the chromosomal origin of expression.

See Example 2 for a description of the methods.

### Example 7

### Detection of microRNA on Beads

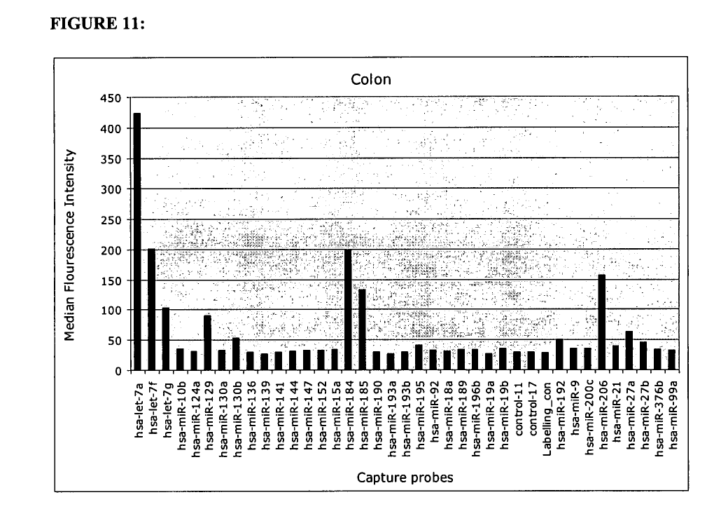

A 37-plex of microspheres coupled with oligonucleotide probes, three of which are control oligonucleotide probes, was used for the analyses of total RNA sample from human colon. The results are shown in FIG. 11. The MFI reading for the negative control, no template control is around 45 (data not shown). The included oligonucleotide probes are shown in Table 3 (validated probes are in bold, the details of which are provided in the subsequent sections):

TABLE 3

| | | | | |
|---|---|---|---|---|
| hsa-let-7a | hsa-miR-130b | hsa-miR-15a | hsa-miR-92 | control-17 |
| hsa-let-7f | hsa-miR-136 | hsa-miR-184 | hsa-miR-18a | labeling_control |
| hsa-let-7g | hsa-miR-139 | hsa-miR-185 | hsa-miR-189 | hsa-miR-192 |
| hsa-miR-10b | hsa-miR-141 | hsa-miR-190 | hsa-miR-196b | hsa-miR-200c |
| hsa-miR-124a | hsa-miR-144 | hsa-miR-193a | hsa-miR-19a | hsa-miR-206 |
| hsa-miR-129 | hsa-miR-147 | hsa-miR-193b | hsa-miR-19b | hsa-miR-376b |
| hsa-miR-130a | hsa-miR-152 | hsa-miR-195 | control-11 | hsa-miR-99a |

### Methods:

Coupling of oligonucleotides. Amino-linked oligonucleotide probes containing LNA were coupled to microspheres according to the standard Luminex protocol.
1. Bring a fresh aliquot of −20° C., desiccated Pierce EDC powder to room temperature.
2. Resuspend the amine-substituted oligonucleotide (“probe”) to 1 mM (1 nanomole/μL) in dH₂O.
3. Resuspend the stock microspheres by vortex and sonication for approximately 20 seconds.
4. Transfer 5.0×10⁶ of the stock microspheres to a microfuge tube.
5. Pellet the stock microspheres by microcentrifugation at ≧8000×g for 1-2 minutes.
6. Remove the supernatant and resuspend the pelleted microspheres in 50 μL of 0.1 M MES, pH 4.5 by vortex and sonication for approximately 20 seconds.
7. Prepare a 1:10 dilution of the 1 mM oligonucleotide probe in dH₂O (0.1 nanomole/μL).
8. Add 2 μL (0.2 nanomole) of the 1:10 diluted oligonucleotide probe to the resuspended microspheres and mix by vortex.
9. Prepare a fresh solution of 10 mg/mL EDC in dH₂O. (Note: Return the EDC powder to desiccant to re-use for the second EDC addition.)
10. One by one for each coupling reaction, add 2.5 μL of fresh 10 mg/mL EDC to the microspheres (25 μg or [0.5 μg/L]_{final}) and mix by vortex.
11. Incubate for 30 minutes at room temperature in the dark.
12. Prepare a second fresh solution of 10 mg/mL EDC in dH₂O. (Note: The aliquot of EDC powder should now be discarded. We recommend using a fresh aliquot of EDC powder for each coupling episode.)
13. One by one for each coupling reaction, add 2.5 μL of fresh 10 mg/mL EDC to the microspheres and mix by vortex.
14. Incubate for 30 minutes at room temperature in the dark.
15. Add 1.0 mL of 0.02% Tween-20 to the coupled microspheres.
16. Pellet the coupled microspheres by microcentrifugation at ≧8000×g for 1-2 minutes.
17. Remove the supernatant and resuspend the coupled microspheres in 1.0 mL of 0.1% SDS by vortex.
18. Pellet the coupled microspheres by microcentrifugation at ≧8000×g for 1-2 minutes.
19. Remove the supernatant and resuspend the coupled microspheres in 100 μL of TE, pH 8.0 by vortex and sonication for approximately 20 seconds.
20. Enumerate the coupled microspheres by hemacytometer:
a. Dilute the resuspended, coupled microspheres 1:100 in dH₂O.
b. Mix thoroughly by vortex.
c. Transfer 10 μL to the hemacytometer, 0.1 mm depth.
d. Count the microspheres within the 4 large corners of the hemacytometer grid.
e. Microspheres/μL=(Sum of microspheres in 4 large corners)×2.5×100 (dilution factor).
21. Store coupled microspheres refrigerated at 2-8° C. in the dark.

The included oligonucleotide probes are shown in the Table 3. Oligo-coupled microspheres were stored at 4° C. until use. For about ⅔ of the oligo-coupled microspheres, synthetic miRNAs were available at Exiqon. These miRNAs (100 fmol) were labeled individually as described below and hybridized to the multiplex to validate coupling of the oligonucleotide probe to the microspheres and to address specificity of the interactions. The validated oligo-coupled microspheres are shown in bold in Table 3.

Labeling. Total RNA samples were labeled using Exiqon labeling kit. In short, a biotin adapter is enzymatically added to available 3′-ends in total RNA. Unincorporated biotin is removed by using a Qiagen RNeasy column. The labeled purified RNA is eluted in water.

Hybridization. Hybridization buffers and conditions are essentially according to the Luminex protocol.
1. Select appropriate oligonucleotide-coupled microsphere sets.
2. Resuspend microspheres by vortex and sonication for approximately 20 seconds.
3. Prepare a Working Microsphere Mixture by diluting coupled microsphere stocks to 150 microspheres of each set/μL in 1.5×TMAC Hybridization Solution.
4. Mix the Working Microsphere Mixture by vortex and sonication for approximately 20 seconds.
5. To each sample or background well, add 33 μL of Working Microsphere Mixture.
6. To each background well, add 17 μL TE, pH 8.
7. To each sample well, add biotinylated complementary oligonucleotide (5 to 200 femtomoles) and TE, pH 8.0 to a total volume of 17 μL.
8. Mix reaction wells gently by pipetting up and down several times.
9. Cover the reaction plate to prevent evaporation and incubate at 95-100° C. for 3 minutes to denature any secondary structure in the sample oligonucleotides.
10. Incubate the reaction plate at 60° C. for 1 hour.
11. Prepare fresh Reporter Mix by diluting streptavidin-R-phycoerythrin to 10 μg/mL in 1×TMAC Hybridization Solution.
12. Add 25 μL of Reporter Mix to each well and mix gently by pipetting up and down several times.
13. Incubate the reaction plate at 60° C. for 15 minutes.
14. Analyze 50 μL at 60° C. on the Luminex analyzer according to the system manual.

### Example 8

### Method for Identification of miRNA Precursors Using Microarrays

A miRNA enriched sample was produced by subjecting 10 mg of human brain total RNA (Ambion FirstChoice® Total RNA) to a fractionation using flashPAGE™ Fractionator and flashPAGE™ Reaction Clean-Up Kit according to the manufacturers instructions. The 30 mL of eluate was concentrated to 10 mL on a Microcon YM-3 Centrifugal Filter Unit (Millipore) according to the manufacturers instructions.

The miRNA enriched sample and 2 mg of human brain total RNA (Ambion FirstChoice® Total RNA) was labeled with Hy3 and Hy5, respectively, using the miRCURY™ LNA Array, Hy3™/Hy5™ labeling kit according to the manufacturers instructions. The two labeling reactions were combined and dried down in a SpeedVac and re-dissolved in 25 mL of RNAse free H₂O. The 25 mL of re-dissolved labeled sample was combined with 25 mL of 2× Hybridization buffer (miRCURY™ LNA Array, 2× hybridization buffer, Exiqon) and hybridized to a miRNA microarray (miRCURY™ LNA Array, ready to spot probe set spotted on CodeLink slides—GE Healthcare and Amersham previously) according to the manufacturers instructions. Hybridization was performed on a hybridization station (Tecan HS4800 Pro system). The hybridization signals were quantified using ImaGene spot analysis software (BioDiscovery) and diagrammed in a scatter plot.

### Example 9

### Detection of Pre-miRNAs on Microarrays

### Microarray Production

Capture probes were designed as reverse complementary sequences to the loop region of 9 human pre-miRNAs in miRBase 9.1 using the same design rules as for capture probes for the mature miRNAs (Table 4). All probes were designed to have a Tm of 72° C. and to include both DNA and LNA nucleotides. These pre-miR capture probes were spotted on microarrays in parallel with the mature miRNA capture probes (miRCURY™ ready-to-spot capture probes version 8.1 (Exiqon)).

Microarrays were spotted on Corning Epoxide slides (Corning) in standard Corning Epoxide spotting buffer at a concentration of 40 μM. Postprocessing of slides was performed according to the slide manufacturer's instructions (Corning).

TABLE 4

| probe | | |
|---|---|---|
| ID | Name | sequence |
| 22274 | premiR_hsa-mir- | aTtgGgAccTgcAcag |
| | 26a-1 | |
| 22275 | premiR_hsa-mir- | cmCtcAcAgAtGgAaAc |
| | 26a-2 | |
| 22276 | premiR_hsa-mir- | aAtGgAgAacAggmCt |
| | 26b | |
| 22201 | premiR_hsa-mir- | gmCacmCttTtmCaaAatmCcamCaa |
| | 15a | |
| 22202 | premiR_hsa-mir- | TcGcaTcTtgActGtAgca |
| | 15b | |
| 22152 | premiR_hsa-mir- | TgAcmCacAaAaTtmCcTtamCac |
| | 10a | |
| 22153 | premiR_hsa-mir- | tgActAtAcgGatAccAcAca |
| | 10b | |
| 22573 | premiR_hsa-mir- | gmCggTccActTcAcc |
| | 99a | |
| 22574 | premiR_hsa-mir- | mCtTgtGtgmCggmCgaa |
| | 99b | |

Sequences of the capture probes targeting the loop region of the human pre-miRNAs used in microarray Example 9. DNA nucleotides are annotated with small letters and LNA nucleotides with capital letters.

### Procedure

4 different RNAs were in vitro-transcribed. These synthetic RNAs had a length of 63-66 nucleotides and their sequence corresponded to 4 different pre-miRNA sequences (pre-hsa-miR-10a, pre-hsa-miR-15a, pre-hsa-miR-26a-1 and pre-hsa-miR-99a as listed in table 5). A mixture of miRCURY™ spike-in kit (Exiqon) for normalization, 1 μg of Ambion yeast total RNA and the 4 pre-miRNAs were labeled with Hy3™ (Exiqon), and a mixture of miRCURY™ spike-in kit and 1 μg of Ambion yeast total RNA were labeled with Hy5™ (Exiqon). The labelling reactions were performed using NEB T4-RNA ligase in a volume of 12.5 μL and following the manufacturers recommendations. The reaction was performed at 20° C. for 1 hour and terminated by 15 minutes at 75° C.

TABLE 5

Sequences of the synthetic pre-miRNAs

| | |
|---|---|
| name | Sequence 5′ to 3′ |

| | |
|---|---|
| pre-hsa-miR-10a | GGGAGAaccctgtagatccgaatttgtgtaaggaattttgtggtcacaaattcgtatctaggggaa |
| pre-hsa-miR-15a | GGGAGAtagcagcacataatggtttgtggattttgaaaaggtgcaggccatattgtgctgcct |
| pre-hsa-miR-26a-1 | GGGAGAttcaagtaatccaggataggctgtgcaggtcccaatgggcctattcttggttacttgca |
| pre-hsa-miR-99a | GGGAGAaacccgtagatccgatcttgtggtgaagtggaccgcacaagctcgcttctatgggtc |

After combining the Hy3™ and Hy5™ labeling reactions, the sample was mixed with 25 μl miRCURY™ hybridisation buffer (Exiqon). The sample was hybridised onto the spotted slide in a Tecan hybridisation machine (Tecan) and hybridised at 60° C. for 16 hours according to standard miRCURY™ array protocols. Washing and drying were also performed in the hybridisation machine according to miRCURY™ array protocol. The experiment was performed in flip-dye.

### Image and Data Analysis

Slides were scanned using an Agilent DNA microarray scanner and images were analysed using Imagene software (BioDiscovery). Each capture probe was spotted 4 times on each microarray, an average of these four measurements on both flip dye slides were used for each probe, and the fold change between the sample with the synthetic miRNAs added and the one without synthetic miRNAs was calculated.

### Results

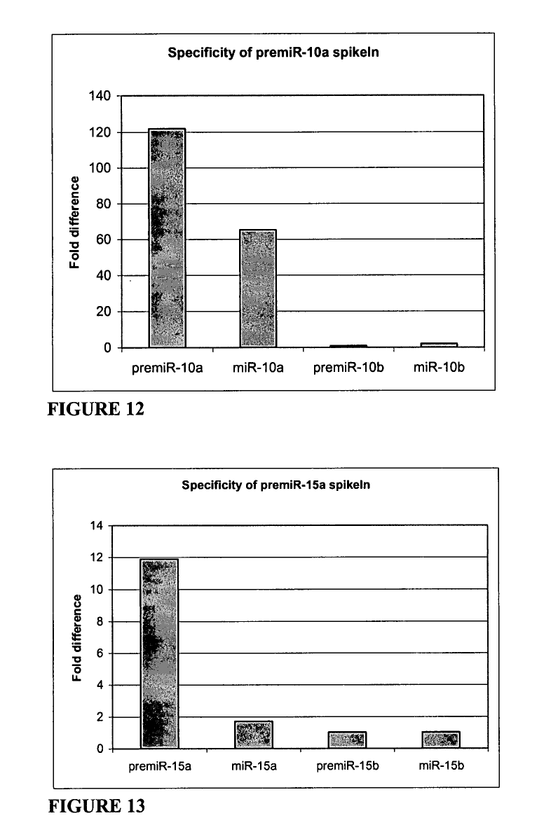

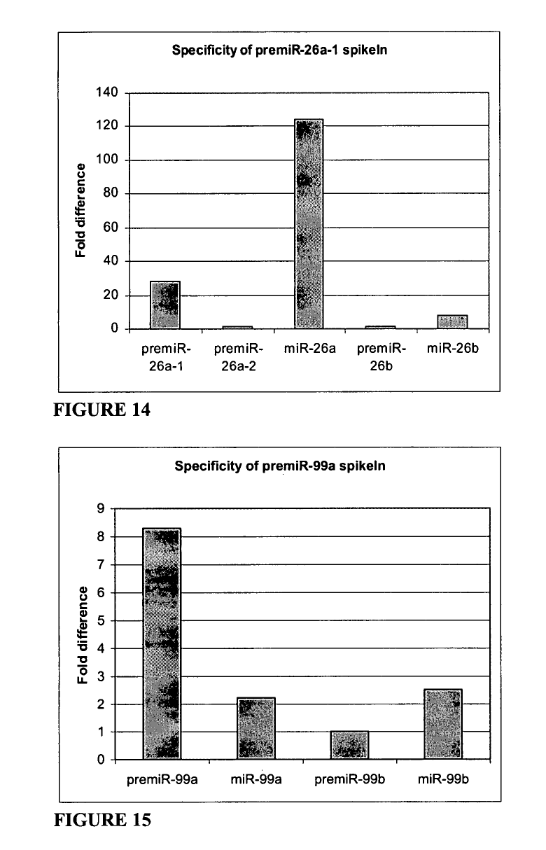

From the analysis of the signals generated from addition of the 4 pre-miRNA, we could conclude that strong signals were generated from both capture probes designed for miRNA and the loop region of the pre-miRNA. In addition, signals from the closely related capture probes were investigated and little/no signals were seen from capture probes with one or more mismatches (FIGS. 12-15). In the case of pre-hsa-mir-26-1 we also investigated if the capture probe for pre-hsa-mir-26-2 gave a signal since it is a precursor for the same miRNA, but no significant signal was observed from this probe (FIG. 14).

### OTHER EMBODIMENTS

All publications, patent applications, and patents mentioned in this specification are herein incorporated by reference.

While the invention has been described in connection with specific embodiments, it will be understood that it is capable of further modifications. Therefore, this application is intended to cover any variations, uses, or adaptations of the invention that follow, in general, the principles of the invention, including departures from the present disclosure that come within known or customary practice within the art.

Other embodiments are within the claims. What we claim is:

1. An oligonucleotide probe that hybridizes to a miRNA precursor molecule, said probe comprising a plurality of nucleotide analogue monomers, wherein at least part of said probe hybridizes to a portion of said precursor not present in the corresponding mature miRNA.

2. The probe of claim 1, wherein said nucleotide analogue monomers are LNA monomers.

3. The probe of claim 1 or 2, wherein at least 50% of said probe hybridizes to said portion of said precursor.

4. The probe of claim 3, wherein at least 70% of said probe hybridizes to said portion of said precursor.

5. The probe of claim 4, wherein at least 80% of said probe hybridizes to said portion of said precursor.

6. The probe of claim 5, wherein at least 90% of said probe hybridizes to said portion of said precursor.

7. The probe of claim 6, wherein all of said probe hybridizes to said portion of said precursor.

8. The probe of claim 1 or 2, wherein said probe does not hybridize to a portion of said mature miRNA.

9. The probe of claim 1 or 2, wherein said probe hybridizes to a portion of said mature miRNA.

10. The probe of claim 1 or 2, wherein said probe hybridizes to 5 nucleotides in the miRNA precursor loop sequence.

11. The probe of claim 1 or 2, wherein said probe hybridizes to the center nucleotide of the miRNA precursor loop sequence.

12. The probe of claim 11, wherein the center nucleotide of said probe hybridizes to the center nucleotide of the miRNA precursor loop sequence.

13. The probe of claim 1 or 2 wherein said plurality of nucleotide analogue monomers hybridize to the loop sequence of the miRNA precursor.

14. The probe of claim 1 or 2 wherein said plurality of nucleotide analogue monomers hybridize to the stem sequence of the miRNA precursor.

15. The probe of claim 1 or 2, wherein two of said plurality of nucleotide analogue monomers are disposed 3 nucleotides apart.

16. The probe of claim 15, wherein only unmodified nucleotides are disposed between said two nucleotide analogue monomers.

17. The probe of claim 15, wherein each of said plurality of nucleotide analogue monomers is disposed 3 nucleotides from the closest nucleotide analogue monomer.

18. The probe of claim 1 or 2, wherein two of said plurality of nucleotide analogue monomers are disposed 4 nucleotides apart.

19. The probe of claim 18, wherein only unmodified nucleotides are disposed between said two nucleotide analogue monomers.

20. The probe of claim 18, wherein each of said plurality of nucleotide analogue monomers is disposed 4 nucleotides from the closest nucleotide analogue monomer.

21. The probe of claim 1 or 2, wherein two of said plurality of nucleotide analogue monomers are disposed adjacent to one another.

22. The probe of claim 21, wherein three of said plurality of nucleotide analogue monomers are disposed adjacent to one another.

23. The probe of claim 22, wherein four of said plurality of nucleotide analogue monomers are disposed adjacent to one another.

24. The probe of claim 21, wherein said plurality is disposed at the 3′ or 5′ end.

25. The probe of claim 21, wherein said plurality is disposed so that one of said nucleotide analogue monomers hybridizes to the center of said miRNA precursor.

26. The probe of claim 1 or 2, comprising at most one mismatched base.

27. The probe of claim 1 or 2, wherein said probe hybridizes to said miRNA precursor under stringent conditions.

28. The probe of claim 27, wherein said probe hybridizes to said miRNA precursor under high stringency conditions.

29. The probe of claim 1 or 2, wherein the melting point of the duplex formed between said probe and said miRNA precursor is at least 1° C. higher than the melting point of the duplex formed between said miRNA precursor and a nucleic acid sequence not comprising a nucleotide analogue monomer.

30. The probe of claim 29, wherein said nucleic acid sequence does not comprise a modified nucleotide.

31. The probe of claim 29, wherein the melting point of the duplex formed between said probe and said miRNA precursor is at least 5° C. higher.

32. The probe of claim 1 or 2, said probe comprising at least 70% DNA.

33. The probe of claim 1 or 2, further comprising a 5′ or 3′ amino group.

34. The probe of claim 1 or 2, further comprising a 5′ or 3′ label.

35. The probe of claim 34, wherein said label is fluorescent.

36. The probe of claim 34, wherein said label is radioactive.

37. The probe of claim 34, wherein said label is targeted by a complex comprising an enzyme.

38. The probe of claim 37, wherein said label comprises digoxigenin (DIG).

39. The probe of claim 35, wherein said label comprises fluorescein.

40. The probe of claim 1 or 2, said probe comprising at least 10% nucleotide analogue monomers.

41. The probe of claim 1 or 2, said probe comprising at most 30% nucleotide analogue monomers.

42. The probe of claim 1 or 2, wherein said probe is at least 15 nucleotides long and at most 30 nucleotides long.

43. A kit comprising a probe of claim 1 or 2 and packaging and labeling indicative of the miRNA precursor to which said probe hybridizes and conditions under which said hybridization occurs.
